(19) United States
(12) Patent Application Publication
(10) Pub. No.: US 2025/0154595 A1
(43) Pub. Date: May 15, 2025

(54) SUBMANDIBULAR GLAND TISSUE BIOMARKER FOR DIAGNOSIS, PROGNOSIS PREDICTION, OR TREATMENT OF PARKINSON'S DISEASE, METHOD FOR DIAGNOSING PARKINSON'S DISEASE, OR PREDICTING PROGNOSIS USING THE SAME, AND METHOD FOR SCREENING SUBSTANCES FOR TREATING PARKINSON'S DISEASE

(71) Applicant: THE CATHOLIC UNIVERSITY OF KOREA INDUSTRY-ACADEMIC COOPERATION FOUNDATION, Seoul (KR)

(72) Inventors: Ko Eun CHOI, Seoul (KR); Joong-Seok KIM, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/941,569

(22) Filed: Nov. 8, 2024

(30) Foreign Application Priority Data
Nov. 9, 2023 (KR) 10-2023-0154871
Jan. 31, 2024 (KR) 10-2024-0015395
Nov. 1, 2024 (KR) 10-2024-0153609

(51) Int. Cl. C12Q 1/6883

(52) U.S. Cl. CPC C12Q 1/6883; C12Q 2600/158; C12Q 2600/178

(57) ABSTRACT

Provided are a submandibular gland tissue biomarker for diagnosis, prognosis prediction, or treatment of Parkinson's disease (PD), a method for diagnosing or predicting the prognosis of PD using the same, and a method for screening substances for treating PD, where it has been confirmed that an SNCAIP gene is significantly decreased and the level of 15 types of miRNA is increased in tissues collected from submandibular gland tissues, and thus PD may be diagnosed using the marker, particularly for subject groups in which the presence or absence of PD cannot be determined by α-synuclein, and since the marker reflects the correlation between genes and environmental factors identified in subjects with PD, the marker can be used as a diagnostic and prognostic biomarker, as well as a new therapeutic target for pathological mechanism of PD.

### BACKGROUND

### Field of the Invention

The present invention relates to a submandibular gland tissue biomarker for diagnosis, prognosis prediction, or treatment of Parkinson's disease (PD), a method for diagnosing PD, or predicting a prognosis using the same, and a method for screening substances for treating PD.

### Discussion of Related Art

PD is a degenerative disease of the central nervous system whose pathophysiological symptoms include deformation of the substantia nigra pars compacta of the midbrain, reduction in brain volume, and aggregation of α-synuclein (αSyn) accompanied by bradykinesia, resting tremors, rigidity and gait disturbance. Abnormal accumulation of phosphorylated αSyn and dopamine deficiency due to dopaminergic neuronal loss in the substantia nigra (SN) are diagnostic markers of PD pathology.

Recent advances in genomic technology have led to the successful identification of a large number of genes that play a role in PD pathogenesis associated with several biological pathways, including αSyn proteolysis, mitochondrial functions, oxidative stress, calcium homeostasis, axonal transport, and neuroinflammation. In particular, it has been reported that frequent point mutations appear in SNCA (PARK1/4), PRKN (PARK2), PINK1 (PARK6), LRRK2 (PARK8), and GBA genes in the case of familial PD.

However, although several large-scale genome-wide association studies have been conducted to discover genes or diagnostic markers associated with sporadic forms of PD, which account for 90% to 95% of all PD patients, no marker that can predict the risk of PD or make a diagnosis with high accuracy has been developed to date.

Meanwhile, the number of PD patients in Korea was 61,565 in 2010, and the number increased to 85,888 in 2014 at an average annual increase rate of 8.7%. In 2014, the proportion of patients aged 60 or older was 95.7%, and the prevalence rate exhibited a correlation with the patient's age. With regard to the proportions according to sex, the patients included 33,831 men and 52,057 women.

Compared to Alzheimer's disease, the onset of PD is much earlier, which is at around 60 years of age. As a result, the progression of the disease continues for 20 years or longer, so there is an urgent need for a treatment method that may slow down the progression of the disease through early detection.

### RELATED ART DOCUMENT

### Patent Document

Korean Patent Publication No. 10-2018-0098153

### SUMMARY OF THE INVENTION

### Technical Problem

An object of the present invention is to provide a method comprising the step of:
(S1) measuring the level of mRNA of an α-synuclein interacting protein (SNCAIP) gene encoding synphilin-1 in a biological sample isolated from a subject;
(S2) comparing the level of the mRNA with the level of the mRNA in a biological sample isolated from a normal control group;
(S3) determining that the subject has Parkinson's disease (PD) when the level of the mRNA in the biological sample isolated from the subject is decreased compared to the level of the mRNA in the biological sample isolated from the control group; and
(S4) treating the PD with chemotherapy, surgery, or biological therapy when PD is diagnosed in step (S3).

Another object of the present invention is to provide a method comprising the step of:
(S1) measuring the level of any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977 in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is increased compared to the level of the miRNA in the biological sample isolated from the control group; and
(S4) treating the PD with chemotherapy, surgery, or biological therapy when PD is diagnosed in step (S3).

Still another object of the present invention is to provide a method comprising the step of:
(S1) measuring the level of any one or more miRNAs selected from the group consisting of hsa-miR-130b-3p, hsa-miR-148a-3p, hsa-miR-301b-3p, hsa-miR-331-3p, hsa-miR-375-3p, hsa-miR-376a-5p, hsa-miR-5690, and hsa-miR-580-3p in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is more than 2-fold change increased compared to the level of the miRNA in the biological sample isolated from the control group, and the AUC value is ≥0.5, the p-value is <0.5, the Confidence Limits value falls between 0.001 and 671; and
(S4) treating the PD with chemotherapy, surgery, or biological therapy when PD is diagnosed in step (S3).

However, the technical problems to be solved by the present invention are not limited to the problems mentioned above, and other problems that are not mentioned can be clearly understood from the description below by those skilled in the art to which the present invention pertains.

### Technical Solution

The present invention provides a method comprising the step of:
(S1) measuring the level of mRNA of an α-synuclein interacting protein (SNCAIP) gene encoding synphilin-1 in a biological sample isolated from a subject;
(S2) comparing the level of the mRNA with the level of the mRNA in a biological sample isolated from a normal control group;
(S3) determining that the subject has Parkinson's disease (PD) when the level of the mRNA in the biological sample isolated from the subject is decreased compared to the level of the mRNA in the biological sample isolated from the control group; and
(S4) treating the PD with chemotherapy, surgery, or biological therapy when PD is diagnosed in step (S3), but not limited thereto.

The present invention provides a method comprising the step of:
(S1) measuring the level of any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977 in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is increased compared to the level of the miRNA in the biological sample isolated from the control group; and
(S4) treating the PD with chemotherapy, surgery, or biological therapy when PD is diagnosed in step (S3), but not limited thereto.

In one embodiment of the present invention, the biological sample may be one selected from the group consisting of submandibular gland tissue, blood, serum, whole blood, plasma, urine, saliva, tissue, cells, organs, bone marrow, a fine needle aspiration specimen, a core needle biopsy specimen, and a vacuum aspiration biopsy specimen, but is not limited thereto.

The present invention provides a method comprising the step of:
(S1) measuring the level of any one or more miRNAs selected from the group consisting of hsa-miR-130b-3p, hsa-miR-148a-3p, hsa-miR-301b-3p, hsa-miR-331-3p, hsa-miR-375-3p, hsa-miR-376a-5p, hsa-miR-5690, and hsa-miR-580-3p in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is more than 2-fold change increased compared to the level of the miRNA in the biological sample isolated from the control group, and the AUC value is >0.5, the p-value is <0.5, the Confidence Limits value falls between 0.001 and 671; and
(S4) treating the PD with chemotherapy, surgery, or biological therapy when PD is diagnosed in step (S3), but not limited thereto.

### Advantageous Effects

According to a submandibular gland tissue biomarker for diagnosis, prognosis prediction, or treatment of PD, a method for diagnosing or predicting the prognosis of PD using the same, and a method for screening substances for treating PD, it was confirmed that the expression level of SNCAIP gene was significantly decreased and the expression level of 15 types of miRNA was increased in tissues collected from submandibular gland tissue. Through the marker according to the present invention, PD may be diagnosed, and in particular, diagnosis may be made for a group in which the presence or absence of PD cannot be determined by α-synuclein gene. In addition, in that the marker of the present invention reflects the correlation between genes and environmental factors identified in subjects with PD, the marker is expected to be used as a diagnostic and prognostic biomarker as well as a new therapeutic target related to the pathological mechanism of PD.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A and 1B show the study workflow and sampling structure for differentially expressed genes (DEGs) and miRNAs (DEmiRNAs), respectively;

FIGS. 2A and 2B are heat maps showing differentially expressed genes (DEGs) and miRNAs (DEmiRNAs), respectively, in submandibular gland (SMG) tissue;

FIGS. 2C and 2D are heat maps showing DEGs and miRNAs (DEmiRNAs), respectively, in blood;

FIGS. 3A to 3D show a top-10 list of gene ontology and Kyoto Encyclopedia of Genes and Genomes (KEGG) pathways as a result of functional annotation and enrichment analysis of DEGs;

FIGS. 3E to 3H show a top-10 list of gene ontology and KEGG pathways as a result of function annotation and enrichment analysis of blood,

FIGS. 3A and 3E show GO; biological processes, FIGS. 3B and 3F show GO; molecular functions; FIGS. 3C and 3G show GO; cellular components, and FIGS. 3D and 3H show KEGG pathways;

FIGS. 4A and 4B show the results of integrated miRNA-mRNA analysis and refer to miRNA in SMG tissue and miRNA in blood, respectively (Here, significant miRNAs were selected when the p-value was less than 0.05. The red line represents a significance threshold value;

FIG. 5A shows the receiver operating characteristic (ROC) curve of has-miR-4449 and area under curves (AUCs);

FIGS. 5B and 5C show the ROC curves of the miRNAs identified in the SMG of the present invention;

FIGS. 5D and 5E show the ROC curves of the miRNAs identified in the blood of the present invention;

FIGS. 6A to 6F show the results of evaluating the correlations of DEGs in the SMG tissue of PD patients with clinical factors that are known to affect the prognosis of PD, including fasting plasma glucose, serum insulin, insulin resistance (homeostasis model assessment of insulin resistance (HOMA-IR) score), physical activity (physical activity scale for the elderly (PASE) score), dietary pattern (Korean version of the Mediterranean diet adherence screener (K-MEDAS) score), and uric acid; and

FIGS. 7A to 7F show the results of evaluating the correlations of DEGs in the blood of PD patients with clinical factors that are known to affect the prognosis of PD, including fasting plasma glucose, serum insulin, insulin resistance (HOMA-IR score), physical activity (PASE score), dietary pattern (K-MEDAS score), and uric acid.

FIGS. 8A and 8B show the interaction network between significant miRNA-mRNA pairs in SMG and FIG. 8C shows the DEmiRNAs interacting with the top 30 hub genes in SMG.

FIGS. 9A and 9B show the interaction network between significant miRNA-mRNA pairs in blood and FIG. 9C shows the DEmiRNAs interacting with the top 30 hub genes in blood.

### DETAILED DESCRIPTION OF EXEMPLARY EMBODIMENTS

The present invention provides a method, including the steps of:
(S1) measuring the level of mRNA of an α-synuclein interacting protein (SNCAIP) gene encoding synphilin-1 in a biological sample isolated from a subject;
(S2) comparing the level of the mRNA with the level of the mRNA in a biological sample isolated from a normal control group;
(S3) determining that the subject has Parkinson's disease (PD) when the level of the mRNA in the biological sample isolated from the subject is decreased compared to the level of the mRNA in the biological sample isolated from the control group; and
(S4) treating the PD with chemotherapy, surgery, or biological therapy when PD is diagnosed in step (S3), but not limited thereto.

The present invention provides a method, including the steps of:
(S1) measuring the level of any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977 in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is increased compared to the level of the miRNA in the biological sample isolated from the control group; and
(S4) treating the PD with chemotherapy, surgery, or biological therapy when PD is diagnosed in step (S3), but not limited thereto.

The present invention provides a method, including the steps of:
(S1) measuring the level of any one or more miRNAs selected from the group consisting of hsa-miR-130b-3p, hsa-miR-148a-3p, hsa-miR-301b-3p, hsa-miR-331-3p, hsa-miR-375-3p, hsa-miR-376a-5p, hsa-miR-5690, and hsa-miR-580-3p in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is more than 2-fold change increased compared to the level of the miRNA in the biological sample isolated from the control group, and the AUC value is >0.5, the p-value is <0.5, the Confidence Limits value falls between 0.001 and 671; and
(S4) treating the PD with chemotherapy, surgery, or biological therapy when PD is diagnosed in step (S3), but not limited thereto.

In step (S4), the treatment of PD includes chemotherapy, surgery, or biological therapy selected from the group consisting of an anti-parkinsonian agent, therapeutic exercise, Deep Brain Stimulation (DBS), or the like, but is not limited to.

In the step of (S3) diagnosing the subject as having Parkinson's disease, the subject may be diagnosed as having Parkinson's disease if the |case/control fold change| is ≥2, preferably if the |case/control fold change| is ≥2.5, and most preferably if the |case/control fold change| is ≥3, but not limited thereto.

In the step of (S3) diagnosing a person as having Parkinson's disease, if the miRNA levels are altered, the subject can be diagnosed as having Parkinson's disease if the AUC value for determining the discriminatory power of the miRNA signature is ≥ 0.5, preferably ≥0.6 or >0.7, most preferably ≥0.8, but not limited thereto.

Further, in the above step of (S3) diagnosing a person as having Parkinson's disease, a subject may be diagnosed as having Parkinson's disease if the p-value is <0.5, preferably <0.4, more preferably <0.3, and most preferably <0.2, but not limited thereto.

Furthermore, during the above steps of (S3) diagnosing a subject as having Parkinson's disease, a person may be diagnosed as having Parkinson's disease if the Confidence Limits value falls between 0.001 and 671, but is not limited thereto.

The present invention provides a composition for diagnosing or predicting the prognosis of PD, including, as an active ingredient, an agent for measuring the expression level of an α-synuclein interacting protein (SNCAIP) gene encoding synphilin-1.

The present invention provides a marker composition for diagnosing or predicting the prognosis of PD, including, as an active ingredient, an SNCAIP gene encoding synphilin-1.

PD is the second most common neurodegenerative disease and primarily affects motor functions, including resting tremors, bradykinesia, rigidity, and loss of postural reflexes. The gold standard for diagnosing PD is confirming the loss of dopaminergic neurons and Lewy body pathology in the substantia nigra within the brain. However, the diagnosis of PD currently depends on clinical characteristics and neuroradiological features because available molecular biomarkers have not been identified. Recent studies have suggested that the progression of Lewy body pathology in PD is the result of intercellular transfer of α-synuclein, which exhibits prion protein-like properties.

The α-synuclein protein is encoded by the SNCA gene located on chromosome 4. The protein is expressed within neurons and generally present in a soluble form. However, in pathological conditions such as PD, α-synuclein aggregates into the form of insoluble fibrils within neurons, thereby forming Lewy bodies. Detecting α-synuclein has been an important topic in the development of molecular biomarkers for PD research. α-Synucleinopathy is widely observed in brain tissues and extracranial tissues such as the submandibular gland (SMG), skin, and colon. Recently, a promising technology using immunoprecipitation-based real-time quaking-induced conversion (IP/RT-QuIC) detected α-synuclein seeds in blood and showed that the structure of α-synuclein fibrils is different in idiopathic PD and multiple system atrophy.

Genome-wide association studies (GWAS) for PD have identified approximately 90 genes associated with the disease. However, most cases of idiopathic PD are not explained by DNA changes detected through GWAS. The GWAS loci identified to date are considered as explaining only about 22% of inherited PD. However, it has been reported that α-synucleinopathy in disease-related tissues such as the SMG accounts for up to 80% of pathological sensitivity. In several previous studies, these pathological changes were investigated in terms of epigenetic changes, such as differences in SNCA RNA expression in the absence of DNA mutations, but the results were controversial.

However, it has been reported that the genetic sequence variations identified in this GWAS analysis may explain only 16% to 36% of the total PD heritability, and the heritability of most of the complex traits that are not explained by GWAS is also referred to as missing heritability.

Although it is true that GWAS has contributed to some extent in establishing the genetic association of PD after the Human Genome Project (HGP), GWAS had limitations of not being able to prove causality, only a small part of the total genetic associations was explained, and the cause of idiopathic PD, which accounts for most PD cases and occurs without a genetic variant, was not properly explained.

In the case of α-synucelinopathy found in the salivary glands of PD patients, it can be confirmed that the pathology sensitivity is reported to be 60% to 80% in different papers, while the SNCA gene association found in GWAS is less than 20%. Therefore, a question obviously arises as to how the α-synucelinopathy known through GWAS occurs, which is not explained by a genetic variant of the SNCA gene that causes α-synucelinopathy in PD and thereby causes damage to dopaminergic neurons, but is clearly confirmed in the salivary gland pathology of PD patients.

In other words, finding a molecular biological method that can explain α-synucelinopathy without an accompanying genetic variant is essential in explaining the pathophysiology of idiopathic PD, which accounts for most PD cases and occurs when specific genetic and environmental factors are unclear, and finding the molecular biomarkers and therapeutic targets.

In other words, the inventors of the present invention confirmed that the expression level of the SNCAIP gene was significantly different even in a group in which the expression level of the α-synuclein gene confirmed in blood or submandibular gland tissue was not differentiated from the control group. In addition, independently, it was confirmed that 15 types of miRNAs were increased at a statistically significant level in the SMG tissue of PD patients compared to the normal control group. In particular, it was confirmed that the 15 types of miRNAs were more statistically significantly capable of diagnosing PD than 14 types of miRNAs in blood. Furthermore, in that the SNCAIP gene and 15 types of miRNAs of the present invention each reflect the characteristics of PD patients that are influenced by environmental factors, it was confirmed that they are biomarkers with better accuracy as they indicate not only gene expression but also the unique characteristics of PD. Therefore, even when this information is described for one of the two kinds of biomarkers of the present invention, it can be equally applied to the other marker.

In one embodiment of the present invention, the composition may be for diagnosing or predicting the prognosis of PD in a group in which the presence or absence of PD cannot be determined by α-synuclein, but is not limited thereto.

In the present invention, “a group in which the presence or absence of PD cannot be determined by α-synuclein” may refer to a group in which the presence or absence of PD cannot be determined by a genetic abnormality in the α-synuclein gene, but is not limited thereto, and it may have a broad meaning that includes all groups in which the presence or absence of PD cannot be determined because the level of α-synuclein is similar to that of a normal control group due to a specific mechanism even when there is no genetic abnormality.

In the present invention, “a group in which the presence or absence of PD cannot be determined” may have a broad meaning that includes not only a group in which the presence or absence of PD cannot be determined by any method known in the art through α-synuclein despite exhibiting the pathological characteristics of PD, but also a group that is not sufficiently diagnosed as having PD but is suspected of having other similar diseases including PD and a group that exhibits no phenotypic/clinical characteristics of PD at all.

In one embodiment of the present invention, the group in which the presence or absence of PD cannot be determined by α-synuclein may have the same expression level of the SNCA gene encoding α-synuclein as that in a normal control group, but is not limited thereto.

In the present invention, “the same expression level as that in a normal control group” may mean including a range that may be considered to be the same as the expression level of the SNCA gene in a normal subject, even if it is not the same numerical value.

In one embodiment of the present invention, the group in which the presence or absence of PD cannot be determined by α-synuclein may have a decreased expression level of the SNCAIP gene encoding synphilin-1, but is not limited thereto.

Therefore, the expression level of the SNCAIP gene confirmed from the SMG tissue of the present invention may be used as a biomarker for PD diagnosis with a higher accuracy for groups in which PD cannot be diagnosed with a genetic abnormality in the α-synuclein gene but PD is suspected, or more precise examination is required.

In particular, in the present invention, although it was confirmed that the α-synuclein expression gene was expressed in the blood or SMG tissue of the PD group at a level that was not significantly different from that of the normal control group, in that it was confirmed that the expression level of the SNCAIP gene encoding synphilin-1 was significantly decreased, and in that it was confirmed that the 15 types of miRNAs identified in the SMG tissue were significantly increased, it was proved that PD may be precisely diagnosed with a higher accuracy even for the groups in which PD is not diagnosed based on the presence or absence of the α-synuclein gene abnormality.

In one embodiment of the present invention, the expression of the gene may be measured from SMG tissue, but is not limited thereto.

The present invention provides a method for providing information for diagnosing or predicting the prognosis of PD, including:
(S1) measuring the level of mRNA of an SNCAIP gene encoding synphilin-1 in a biological sample isolated from a subject; and
(S2) comparing the level of the mRNA with the level of the mRNA in a biological sample isolated from a normal control group.

In one embodiment of the present invention, the method for providing information may further include (S3): determining that the subject has PD when the level of the mRNA in the biological sample isolated from the subject is decreased compared to the level of the mRNA in the biological sample isolated from the control group, but is not limited thereto.

In the present invention, a “biological sample” may include anything without limitation as long as it is taken from a subject for whom PD is to be diagnosed or the prognosis thereof is to be predicted.

In one embodiment of the present invention, the biological sample may be one selected from the group consisting of an SMG tissue, blood, serum, whole blood, plasma, urine, saliva, tissue, cells, organs, bone marrow, a fine needle aspiration specimen, a core needle biopsy specimen, and a vacuum aspiration biopsy specimen, but is not limited thereto.

The biological sample may be pretreated before it is to be used for detection or diagnosis. For example, the pretreatment may include homogenization, filtration, distillation, extraction, concentration, inactivation of interfering ingredients, and addition of reagents, etc. The sample may be prepared to increase the detection sensitivity of a protein marker, for example, a sample obtained from a subject may be pretreated using methods such as anion exchange chromatography, affinity chromatography, size exclusion chromatography, liquid chromatography, sequential extraction or gel electrophoresis.

The term “method for providing information” used in the present invention refers to a method of providing information regarding the diagnosis of a disease, and it refers to a method of acquiring information about the onset or possibility (risk) of developing a disease by analyzing a biological sample of a subject or confirming an increase or decrease in the level of a biomarker according to the present invention. For example, the method for providing information is a method of measuring the level of a biomarker according to the present invention and comparing it with a control group to provide information on whether to diagnose an subject as having PD or whether the prognosis prediction is good or bad, and through the method, by means of appropriate treatment for PD, it is possible to diagnose PD especially in groups in which PD is not diagnosed based on the presence or absence of α-synuclein gene abnormalities, prescribe the necessary treatment method, and predict the therapeutic effect of the treatment, through appropriate treatment for PD.

In the present specification, the expression “the level is increased” means that something that was not detected has been detected, or that the amount of detection is relatively higher than the normal level. For example, an “increased” level means that the level in the experimental group is at least 1%, 2%, 3%, 4%, 5%, 10% or more, for example, 5%, 10%, 20%, 30%, 40%, or 50%, 60%, 70%, 80%, 90% or higher than that of the control group, and/or 0.5 times, 1.1 times, 1.2 times, 1.4 times, 1.6 times, 1.8 times or higher that of the control group. Specifically, it may mean that the level is higher than that of the control group by 1 to 1.5 times, 1.5 to 2 times, 2 to 2.5 times, 2.5 to 3 times, 3 to 3.5 times, 3.5 to 4 times, 4 to 4.5 times, 4.5 to 5 times, 5 to 5.5 times, 5.5 to 6 times, 6 to 6.5 times, 6.5 to 7 times, 7 to 7.5 times, 7.5 to 8 times, 8 to 8.5 times, 8.5 to 9 times, 9 to 9.5 times, 9.5 to 10 times, or 10 times or more that of the control group, but is not limited thereto. In addition, in the present invention, the expression that “the expression level of the SNCAIP gene is decreased” or “the level of 15 types of miRNAs identified in SMG tissue is increased” may have a broad meaning including a statistically significant level, but is not limited to thereto. The meaning of the opposite term may be understood by those skilled in the art as having the opposite meaning according to the above definition.

In the present invention, when the term “including” is used, unless otherwise specified, it means that other components may be further included rather than excluding other components. The term “step of . . . ” used throughout the present invention does not mean “step for.”

The present invention provides a method for screening substances for preventing or treating PD, including a step of measuring the level of mRNA of an SNCAIP gene encoding synphilin-1 in a biological sample isolated from a PD animal model administered a candidate substance.

In one embodiment of the present invention, the screening method may further include a step of selecting the candidate substance as a substance for preventing or treating PD when the level of the mRNA is increased in a biological sample isolated from a subject, but is not limited thereto.

According to an embodiment of the present invention, it was confirmed that the SNCAIP gene or the 15 types of miRNAs identified in the SMG tissue of the present invention are not only biomarkers for diagnosing or predicting the prognosis of PD but also targets for treatment, that is, therapeutic biomarkers of PD. Therefore, when developing a therapeutic agent for PD, an agent capable of regulating (reducing) the expression level of the SNCAIP gene of the present invention may be confirmed to be a therapeutic substance for PD through an in vivo or in vitro experiment. In particular, it was confirmed that the SNCAIP gene or the 15 types of miRNAs identified in the SMG tissue of the present invention may be used in diagnosing or predicting the prognosis of PD in groups in which the presence or absence of PD cannot be determined based on α-synuclein gene abnormalities. As described above, “a group in which the presence or absence of PD cannot be determined” includes both the groups that are not sufficiently diagnosed as having PD but are suspected of having PD and the groups that exhibit no phenotypic/clinical characteristics of PD at all. Therefore, the markers may be used as excellent therapeutic markers in treating a group in which the presence or absence of PD cannot be determined based on the conventional α-synuclein with a therapeutic substance and then confirming the increase/decrease of the SNCAIP gene or the 15 types of miRNAs identified in the SMG tissue of the present invention to confirm that the substance is a therapeutic substance for PD.

In the present invention, “screening” may refer to selecting a substance with specific properties from a candidate group consisting of various substances using a specific manipulation or evaluation method.

In the present invention, “candidate substance” refers to an unknown substance used in screening by administering to a PD animal model to measure the increase/decrease in expression of the SNCAIP gene or the 15 types of miRNAs of the present invention, and it may be one or more selected from the group consisting nucleotides, DNA, RNA, amino acids, aptamers, proteins, stem cells, stem cell culture solutions, compounds, microbial cultures or extracts, natural products, and natural extracts, but is not limited thereto.

In the present invention, the term “treatment” refers to all actions that improve or beneficially change a target disease and metabolic abnormalities thereof, and methods such as chemotherapy, surgery, or biological therapy may be used.

In the present invention, although substances or methods that are commonly used in the art to treat PD are applied, additionally, commonly used treatment methods may be used, or drugs that are commonly used to assist in the treatment of PD may further be administered, and the candidate substances disclosed in the present invention may be administered, but are not limited thereto.

In the present invention, a method of measuring an mRNA level or miRNA level is not particularly limited as long as the method is an mRNA measurement method known in the art, but it may be measured by methods such as polymerase chain reaction (PCR), RNase protection assay, northern blotting, southern blotting, in situ hybridization, a DNA chip, and/or an RNA chip, next-generation sequence (NGS), including total RNA sequencing and small RNA sequencing, but the method is not limited thereto and may include all methods commonly used in the art.

The present invention provides a kit for diagnosing or predicting the prognosis of PD, including an agent for measuring the expression level of an SNCAIP gene encoding synphilin-1; and instructions.

In the present invention, “kit” refers to a tool further including substances for preparation or function, storage, or the like that enable the purpose of kit claimed in the present invention to be performed. In addition to the above substances, the kit of the present invention may include other components, compositions, solutions, devices, and the like commonly required for storage and processing methods. As a specific example, at this time, each component may be applied one or more times without limit, there is no limit in the sequence of application of each material, and the application of each material may be carried out simultaneously or at different times.

In the present invention, the kit may include a container; instructions; or the like. The container may serve to package the substances, and may also serve to store and secure them. The material of the container may be in the form of, for example, a bottle, a tub, a sachet, an envelope, a tube, an ampoule, or the like, which may be partially or entirely made of plastic, glass, paper, foil, wax, or the like. The container may be equipped with a completely or partially removable cover that may initially be part of the container or may be attached to the container by mechanical, adhesive, or other means, or a stopper that may provide access to the contents by a needle may be mounted. The kit may include an external package, and the external package may include instructions for use of the components. In addition, the same information may be applied to the 15 types of miRNAs of the present invention.

The present invention provides a composition for diagnosing or predicting the prognosis of PD, including, as an active ingredient, an agent for measuring the level of any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977.

The present invention provides a marker composition for diagnosing or predicting the prognosis of PD, including, as an active ingredient, any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977.

In one embodiment of the present invention, the miRNA may be measured from submandibular gland tissue, but is not limited thereto.

The present invention provides a method for providing information for diagnosing or predicting the prognosis of PD, including:
(S1) measuring the level of any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977 in a biological sample isolated from a subject; and
(S2) comparing the level of the miRNA with the level of the miRNA in a biological sample isolated from a normal control group.

In one embodiment of the present invention, the method for providing information may further include (S3): diagnosing the subject as having PD when the level of the miRNA in the biological sample isolated from the subject is increased compared to the level of the miRNA in the biological sample isolated from the control group, but is not limited thereto.

In one embodiment of the present invention, the biological sample may be one selected from the group consisting of submandibular gland tissue, blood, serum, whole blood, plasma, urine, saliva, tissue, cells, organs, bone marrow, a fine needle aspiration specimen, a core needle biopsy specimen, and a vacuum aspiration biopsy specimen, but is not limited thereto.

The present invention provides a method for screening substances for preventing or treating PD, including a step of measuring the level of any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977 in a biological sample isolated from a PD animal model administered a candidate substance.

In one embodiment of the present invention, the screening method may further include a step of selecting the candidate substance as a substance for preventing or treating PD when the level of the miRNA is decreased in a biological sample isolated from a subject, but is not limited thereto.

According to an embodiment of the present invention, it was confirmed that the 15 types of miRNAs of the present invention are not only biomarkers for diagnosing or predicting the prognosis of PD but also targets for treatment, that is, therapeutic biomarkers of PD. Therefore, when developing a therapeutic agent for PD, an agent capable of regulating (reducing) the level of the 15 types of miRNAs of the present invention may be confirmed to be a therapeutic substance for PD through an in vivo or in vitro experiment.

The present invention provides a kit for diagnosing or predicting the prognosis of PD, including an agent for measuring the level of any one or more miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977; and instructions.

In addition, the present invention may provide a diagnostic device for diagnosing, predicting a prognosis, and even treating PD in a subject. A measurement unit of the diagnostic device of the present invention may use an agent for measuring the expression level of the SNCAIP gene or the levels of the 15 types of miRNAs, which are biomarkers according to the present invention, to measure the expression level of a protein or gene in a biological sample (e.g., blood, etc.) obtained from a subject. By measuring the expression level of the protein or gene using the agent in the measurement unit, PD may be diagnosed, or the treatment may be diagnosed as having a high treatment effect.

The diagnostic device of the present invention may further include a detection unit that predicts and outputs the presence, disease stage, or type of PD in a subject based on the expression level of the protein or gene obtained in the measurement unit.

In the present invention, the detection unit may determine the implementation of the combination treatment by generating and classifying information on the diagnosis or prognosis prediction of PD according to the category of the expression level of the protein or gene obtained in the measurement unit.

In the present invention, a “biomarker” may refer to a marker that can distinguish between normal and pathological conditions, predict treatment responses and be objectively measurable.

In the present invention, “diagnosis” refers to confirming the presence or characteristics of a pathological condition. For the purpose of the present invention, diagnosis is not limited to confirming the diagnosis of PD, but it also includes predicting the prognosis of PD.

More specifically, the term used in the present invention, “diagnosis,” refers to determining the susceptibility of a subject to a specific disease or disorder, determining whether a subject currently has a specific disease or disorder, determining the prognosis (e.g., identifying a tumor state, determining the stage of a tumor, or determining the responsiveness of cancer to treatment, in the present invention, diagnosis of PD or progression of the disease) of a subject with a specific disease or disorder, or therametrics (e.g., monitoring the conditions of a subject to provide information about treatment efficacy).

In the present invention, “measurement” has a meaning including detecting and confirming the presence (expression) of a target substance, or detecting and confirming a change in the level of presence (expression level) of a target substance. The measurement may be performed without limitation and includes both qualitative methods (analysis) and quantitative methods. The types of qualitative methods and quantitative methods for measuring the presence/level of a biomarker of the present invention are well known in the art and include the experimental methods described in the present specification.

The term “analysis” used in the present specification may preferably refer to “measurement,” and the qualitative analysis may refer to measuring and confirming the presence of a target substance, and the quantitative analysis may refer to measuring and confirming a change in the level of presence (expression level) or amount of a target substance. In the present invention, analysis or measurement may be performed without limitation and includes both qualitative methods and quantitative methods, and quantitative measurement may be preferably performed.

In the present invention, “prognosis prediction” may refer to predicting the degree of disease progression when a subject diagnosed as having a disease is treated with a specific therapeutic substance. In other words, in the present invention, it may mean predicting the probability of progression, worsening, recurrence, maintenance, etc. of PD through the increase or decrease in the level of a biomarker of the present invention.

In the present invention, “subject” means a target that requires disease risk prediction, diagnosis, prognosis prediction, or treatment, and more specifically, it may refer to human or non-human primates, mammals such as mice, rats, dogs, cats, horses, and cows, but is not limited thereto.

In the present invention, “determination” may be used interchangeably with “discrimination” and refers to distinguishing a subject according to a specific standard. In the present invention, it may be used to refer to distinguishing whether a subject diagnosed as having or likely to have PD will show a therapeutic effect to a PD therapeutic agent, or distinguishing those who have sensitivity to the above combination of treatments from those who do not, but is not limited thereto.

In the present invention, “administration” refers to providing a predetermined composition of the present invention to a subject by any appropriate method.

In the present invention, “prevention” refers to all actions that suppress or delay the onset of a target disease, and “treatment” refers to all actions that improve or beneficially change a target disease and metabolic abnormalities thereof by administration of a therapeutic substance, and “improvement” refers to all actions that reduce parameters related to a target disease, such as the degree of symptoms, by administration of a therapeutic substance.

Hereinafter, preferred examples are presented to aid understanding of the present invention. However, the following examples are provided only to make the present invention easier to understand, and the content of the present invention is not limited by the following examples.

### EXAMPLES

### Participants

The study protocol was approved by the Institutional Review Board of the Catholic University of Korea Seoul St. Mary's Hospital (IRB approval number KC22TISI0415). All participants provided written consent prior to their participation.

This study included seven patients newly diagnosed with PD according to the UK Brain Bank criteria (24) and a control group consisting of six subjects who underwent SMG resection for head and neck cancer at the Department of Neurology and the Department of Otolaryngology-Head and Neck Surgery at the Catholic University of Korea Seoul St. Mary's Hospital between October 2022 and April 2023 without an age-related, gender-related, neurological or psychological disorder. The patient exclusion criteria were as follows: (1) young onset PD (age of onset of 40 years or less), (2) family history of PD, (3) normal dopamine transporter scan according to Movement Disorder Society's clinical diagnostic criteria for PD, (4) neurological or neuroradiological abnormalities associated with atypical PD or secondary PD, (5) abnormalities of the basal ganglia or cerebellum found in structural magnetic resonance imaging, (6) use of an anticoagulant, (7) known SMG disease, (8) elderly people (people over 75 years of age who may be vulnerable to invasive procedures), and (9) severe dementia.

Medical record review and neurological examination were performed on the control subjects to ensure that each subject was free from neurological or psychological disorders, including idiopathic rapid eye movement (REM) sleep behavior disorder, essential tremor, PD, dementia, and depression.

Clinical information on age, gender, body mass index (BMI), hypertension, diabetes, smoking habits, and dietary habits was collected using the K-MEDAS questionnaire, and information on physical activity habits was collected using the PASE questionnaire for the PD patients and controls. In addition, disease duration, a Unified Parkinson's disease Rating Scale (UParkinsonRS) score, and a modified Hoehn and Yahr (H&Y) staging score of the PD patients were determined.

### Sample Collection Procedure

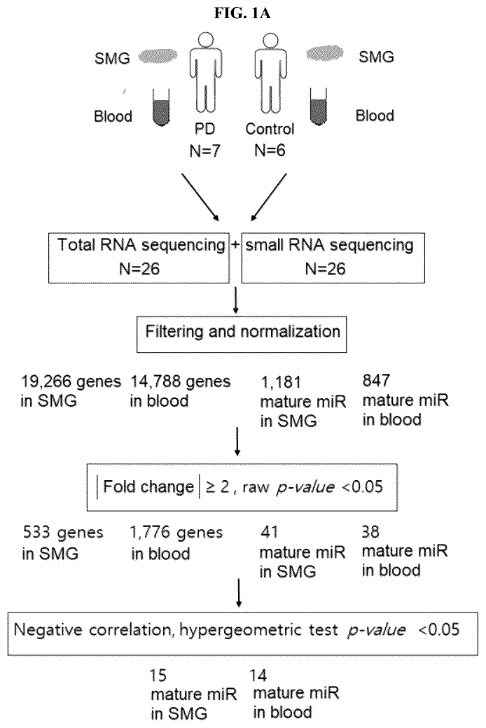

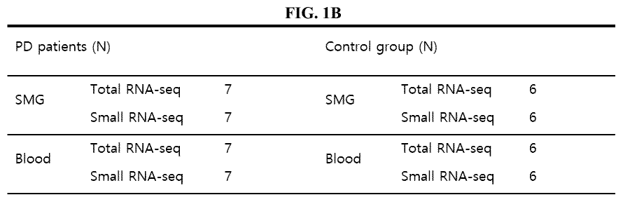

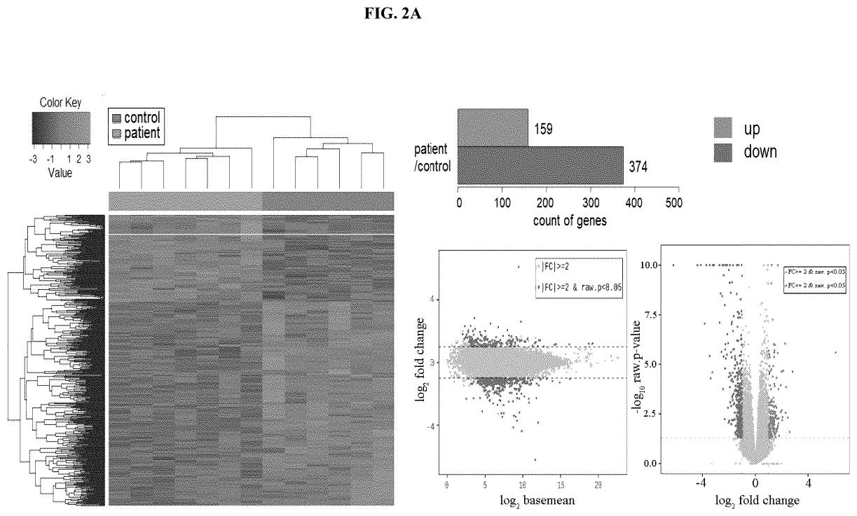

The study workflow is shown in FIG. 1A. SMG tissue and blood samples were obtained from each patient on the same day. Ultrasound-guided core needle biopsy (CNB) was performed by an experienced radiologist to obtain the patient SMG tissue. After subcutaneous local anesthesia with 1% lidocaine, the operator attempted to obtain the SMG tissue using a disposable 18-gauge needle core biopsy instrument kit (Bard Mission Disposable Core Biopsy Instrument kit, Beckton, Dickinson, and Company, Franklin Lakes, NJ, USA) for up to two examinations. All patients were continuously monitored by real-time ultrasound to check for complications such as bleeding at the biopsy site and hematoma in the SMG tissue. For all patients, local compression was applied to the biopsy site for 20 to 30 minutes. Control SMG tissue was obtained from surgical specimens resected during head and neck cancer surgery, such as modified radical neck dissection (MRND). The size of the obtained SMG tissue was approximately 1.2 mm in diameter and approximately 7 to 10 mm in length in the CNB in the PD patients and an approximately 3 mm cubic size in the controls. All tissues were immersed in RNAlater™ Stabilization Solution in RNAlater Tissue Protect Tubes (Thermo Fisher Scientific, Waltham, MA) immediately after the procedure to prevent RNA degradation. The obtained SMG tissue was divided into two pieces for transcriptome sequencing and smRNA-seq. The sampling ischemia time was less than one minute for the PD patients and 1 to 20 minutes for the controls.

Blood samples were collected on the same day of the SMG procedure. Approximately 3 mL of whole blood was obtained from each PAXgene Blood RNA Tube, and a total of 2 PAXgene Blood RNA Tubes were collected from each participant. One tube was for transcriptome sequencing, and the other tube was for smRNA-seq. Clinical blood samples were collected simultaneously to measure fasting plasma glucose, serum insulin, hemoglobin A1c (HbA1c), C-peptide, and lipid profiles for the PD patients and controls. Fasting was defined as abstinence from caloric intake for at least eight hours.

### Sample Preparation and RNA-Seq

RNA-seq was performed on the SMG tissue and blood samples (n=52; 26 total RNA, 26 smRNA) from all the study participants (FIGS. 1A and 1B). RNA was extracted from the SMG tissue using the QIAzol lysis reagent according to the Trizol tissue RNA extraction protocol, and RNA was extracted from the blood samples according to the PAXgene Blood RNA kit instructions. All samples had an RNA integrity number (RIN)≥6 except for one blood sample in which RIN=4.9 (mean RIN=7.88, range of 4.9 to 8.8). All samples were included for cDNA library construction.

The libraries were prepared using the Illumina TruSeq Standard Total RNA Library Prep Gold Kit (Illumina, Inc., San Diego, CA, USA) for the SMG tissue, the Illumina TruSeq Standard Total RNA Library Prep Globin Kit (Illumina, Inc.) for the blood, and the Illumina TruSeq Small RNA Library Prep Kit (Illumina, Inc.) for both the SMG tissue and blood samples. The total RNA library was validated using TapeStation D1000 ScreenTape (Agilent Technologies, Santa Clara, USA), and the smRNA library was assessed for size, purity, and concentration using an Agilent Bioanalyzer (Agilent Technologies). A validated index library was submitted for sequencing on the Illumina NovaSeq™ 6000 (Illumina, Inc.). Paired-end (2×100 bp) sequencing was performed on the total RNA-seq library for transcriptome analysis, and single-end (51 bp) sequencing was performed on the smRNA-seq library for miRNA analysis.

### RNA-Seq Data Analysis

As a computational analysis method, raw read sequencing data (FASTQ) was applied for quality assessment of reads, trimming, alignment, normalization, expression, and functional annotation. For total RNA-seq, 3′ adapter sequences and low-quality reads were removed using Trimomatic 0.38 (http://www.usadellab.org/cms/?page=trimmomatic). Trimmed reads were mapped to the human GRCh38 (hg38) reference genome (annotation NCBI_109.20200522) using HISAT2 (https://ccb.jhu.edu/software/hisat2/index.shtml). Expression profiling was performed with the mapped reads using String Tie (https://ccb.jhu.edu/software/stringtie/). For each gene, fragments per kilobase of transcripts per million (FPKM) and transcripts per million (TPM) were obtained. After filtering out underexpressed genes using one or more zero counts, the remaining reads were normalized using the Relative Log Expression (REL) method implemented in the DEseq2 package in the R library (http://www.bioconductor.org/). Differentially expressed genes between the PD patients and controls were estimated by the negative binomial Wald test using DEseq2. Criteria for significant results were |case/control fold change|≥2 and raw p-value<0.05.

Gene ontology (GO) enrichment analysis and functional annotation were performed on differentially expressed genes (DEGs) using the g: Profiler tool (https://biit.cs.ut.ee/gprofiler/). The relevant biological pathways associated with the DEGs were analyzed using the Kyoto Encyclopedia of Genes and Genomes (KEGG) pathway database (http://www.kegg.jp/kegg/pathway.html). For smRNA-seq, Cutadapt 2.8 (https://cutadapt.readthedocs.org/en/stable/) was used for trimming. The finally processed reads were sequentially aligned to the reference genome, miRBase v22.1 (http://www.mirbase.org/). Genome mapping was processed with Bowtie (http://bowtie-bio.sourceforge.net/index.shtml) and Spliced Transcripts Alignment to a Reference (STAR) using RNA-Seq by Expectation-Maximization (RSEM) (http://deweylab.github.io/RSEM/). Known/novel miRNAs were predicted using miRDEEP2 (https://www.mdc-berlin.de/content/mirdeep2-documentation). After excluding mature miRNAs with zero counts across more than 51% of all samples, read count data was normalized using a trimmed average M-value (TMM) method in the edgeR library (https://bioconductor.org/packages/3.17/bioc/). Differentially expressed miRNAs were analyzed by an exact test using edgeR, and significance was determined based on |case/control fold change|≥2 and raw p-value<0.05.

Hierarchical clustering through Euclidean distance and complete linkage grouped the similarities of the genes and the mature miRNAs based on the normalized expression levels of the significant list. miRNA-mRNA integration analysis extracted the differentially expressed mRNAs and miRNAs between the compared samples, and a negative correlation of the target pairs between mRNAs and miRNAs was confirmed based on miRDB v6.0 (http://mirdb.org/miRDB/). The p-value was derived from a hypergeometric test for the mRNA targeted by the miRNA. The sample preparation, next-generation sequencing (NGS), and computational sequencing analysis were performed at Macrogen Inc. (Seoul, Korea).

### Statistical Analysis

Descriptive statistics were presented as mean±standard deviation (SD) or frequency for each clinical characteristic. Analysis was performed using the independent t-test for continuous variables and the Fisher's exact test for categorical variables. Pearson's correlation coefficient was used to determine the correlation between DEGs and clinical factors, including fasting plasma glucose, serum insulin, HOMA-IR score, serum uric acid, K-MEDAS score, and PASE score. As the normalized expression values of DEGs, the TPM value for each gene was used. For the PD patients, the covariates of age, gender, BMI, and disease duration were controlled, and for the control group, age, gender, and BMI were controlled.

A binomial logistic regression model was fit to investigate the diagnostic ability of the identified miRNAs based on an integrated analysis of miRNAs, excluding has-miR-12136 in the blood due to complete separation. The predicted probability was calculated based on receiver-operating characteristic (ROC) curve and area under the curve (AUC) analysis, excluding has-miR-12136 in the blood. The normalized expression values for each miRNA were calculated using TMM. The difference between multiple ROC curves and AUC values of miRNAs was compared using Wilcoxon-Mann-Whitney U statistics.

All clinical statistical analyses were performed using SAS (version 9.4; SAS Institute Inc., Cary, NC, USA) statistical software. A p-value <0.05 was considered statistically significant.

Construction of the protein-protein interaction (PPI) network analysis was performed using the Search Tool for the Retrieval of Interaction Genes (STRING) database (version 12.0) (http://www.string-db.org/) with confidence threshold of 0.2. Subsequently, the hub genes in the interaction network were identified by analyzing the degree of connectivity using Cytoscape (version 3.10.2) and the cytoHubba plugin (version 0.1). To visualize the interaction network of target genes with differentially expressed miRNAs (DEmiRNAs), Cytoscape (version 3.10.2) was also employed.

### Example 1. Clinical Characteristics

A total of 52 samples were collected from seven PD patients and six controls. Of these samples, 28 were from the PD patients and 24 were from the controls (FIGS. 1A and 1B). The clinical characteristics of the PD patients and the controls are summarized in Table 1. The patients and controls recruited for this study were matched for age (PD patients vs. controls; 64.1±6.1 vs. 61.7±6.1, p=0.4792) and gender (p=1). There was no significant difference between the PD patients and the controls in the baseline characteristics of BMI, smoking history, hypertension, diabetes, fasting plasma glucose, glycated hemoglobin (HbA1c), serum insulin, C-peptide, HOMA-IR score, uric acid, and K-MEDAS score. The mean disease duration of the PD patients was 17±14.0 months, and a significant difference was observed in the physical activity score between the PD patients and controls (PASE score, PD patients vs. controls, 204.2±60.8 vs. 95.9±49.6; p=0.0046). Procedure-related complications were carefully monitored and found to be mild, controllable, and well-tolerated (Table 2, Procedure-related Complication Profile).

TABLE 1

| Item | Patients | | Controls | | p-value |
|---|---|---|---|---|---|
| N | 7 | | 6 | | |
| Age, year | 64.1 ± 6.1 | | 61.7 ± 6.1 | | 0.4792 |
| Sex, n (%) | | | | | 1.0000 |
| Men | 4 | (57.1%) | 3 | (50%) | |
| Women | 3 | (42.9%) | 3 | (50%) | |
| BMI, kg/m² | 24.9 ± 3.8 | | 24.7 ± 6.5 | | 0.9475 |
| Smoker, n (%) | | | | | 0.5594 |
| Non-smoker | 4 | (57.1%) | 5 | (83.3%) | |
| Ex-smoker | 3 | (42.9%) | 1 | (16.7%) | |
| Hypertension, n (%) | 2 | (28.6%) | 4 | (66.7%) | 0.2861 |
| Diabetes mellitus, n (%) | 0 | (0%) | 2 | (33.3%) | 0.1923 |
| Fasting plasma glucose | 106 ± 15.9 | | 135.8 ± 34.7 | | 0.0649 |
| (mg/dL) | | | | | |

| | | | |
|---|---|---|---|
| HbA1c(%) | 5.7 ± 0.2 | 5.9 ± 0.5 | 0.3341 |
| Insulin (uIU/mL) | 10.1 ± 7.6 | 7.42 ± 4.1 | 0.4604 |
| C-peptide(ng/mL) | 2.1 ± 0.96 | 2.3 ± 0.68 | 0.5999 |
| HOMA-IR score | 2.6 ± 1.9 | 2.4 ± 1.3 | 0.8042 |
| Uric acid (mg/dL) | 4.6 ± 1.3 | 4.7 ± 1.4 | 0.8501 |
| Disease Duration, month | 17 ± 14.0 | N/A | |
| UPDRS part III | 30.4 ± 18.6 | N/A | |
| UPDRS total | 42.1 ± 20.4 | N/A | |
| H & Y stage | 1.9 ± 1.1 | N/A | |
| K-MEDAS score | 7.6 ± 2.1 | 6.8 ± 3.4 | 0.6416 |
| PASE score | 95.9 ± 49.6 | 204.2 ± 60.8 | 0.0046* |

TABLE 2

| | Number of | |
|---|---|---|
| Complications | patients | Description |
| Pain | 2 | Temporary, mild pain; relieved |
| | | spontaneously without analgesics |
| Puncture site | 1 | Minimal bleeding; Controlled by 20 min |
| bleeding | | ice-pack compression. |
| Hematoma in | 1 | Mild hematoma (<0.5 cm in diameter); |
| SMG | | Ultrasound confirmed bleeding control after |
| | | 20 min ice-pack compression. |
| Infection | 0 | Did not occur. |

### Example 2. Differential Expression of mRNA and miRNA in PD Patients

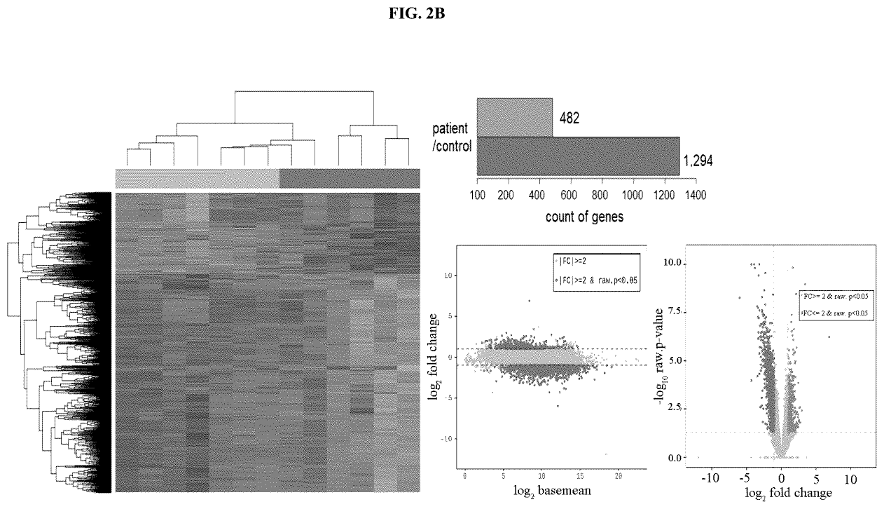

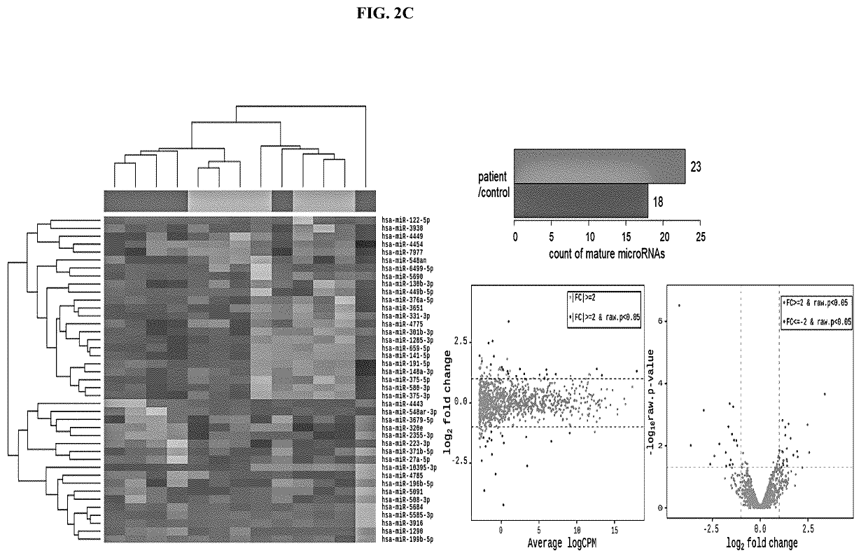

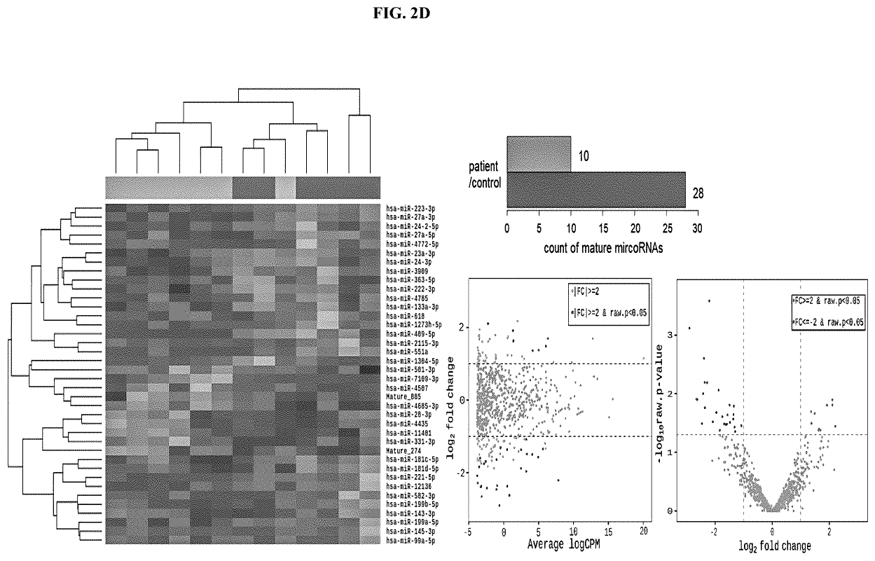

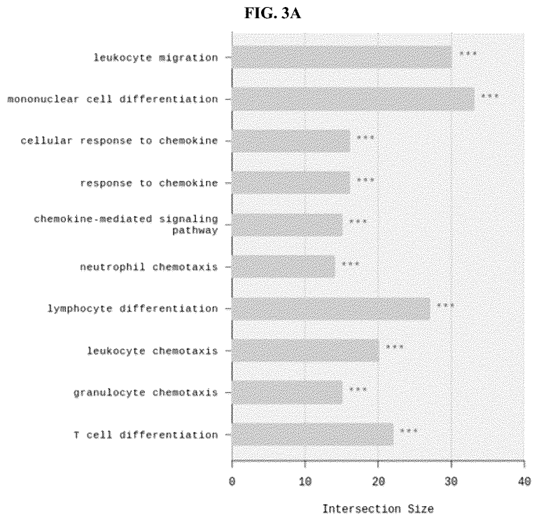

The whole transcriptome sequencing data and smRNA-seq data were deposited in the National Center for Biotechnology Information (NCBI) (Sequence Read Archive SRA) database (BioProject accession number: PRJNA1027508). After filtering out underexpressed genes and miRNAs, a total of 19,266 genes in the SMG tissue, 14,788 genes in the blood, 1,181 mature miRNAs in the SMG tissue, and 847 mature miRNAs in the blood were processed for differential expression analysis. A total of 533 genes and 41 mature miRNAs were found in the SMG tissue, and 1,776 genes and 38 miRNAs in the blood showed significant differential expression in the PD patients (FIG. 1A). Heatmap visualization shows the up-regulated and down-regulated differentially expressed genes (DEGs) (FIGS. 2A and 2B) and the differentially expressed miRNAs (FIGS. 2C and 2D), and the dendrogram shows the hierarchical clusters.

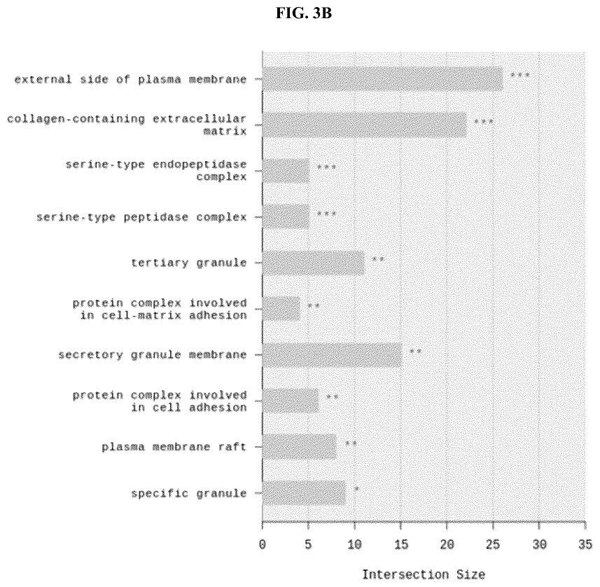

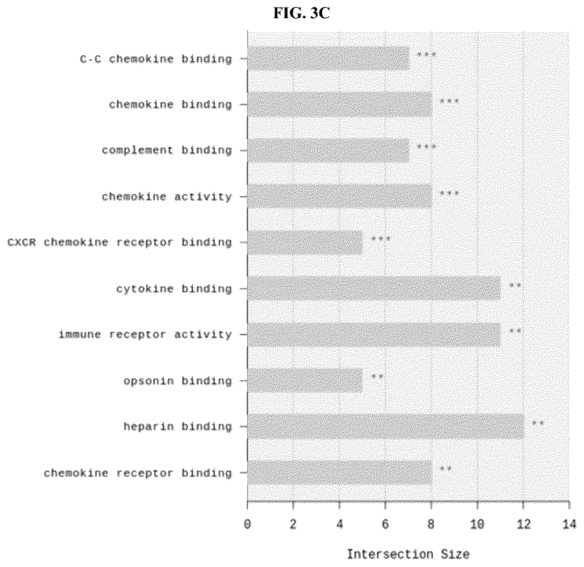

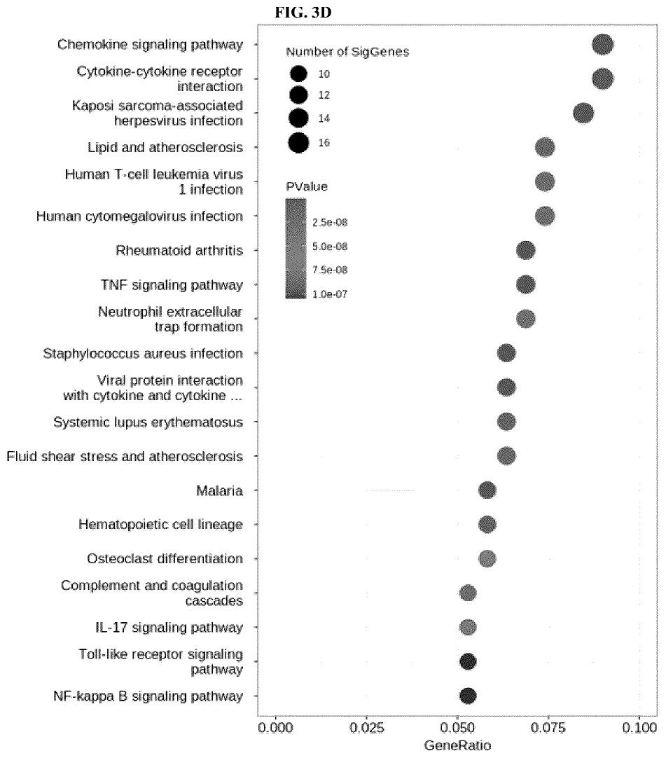

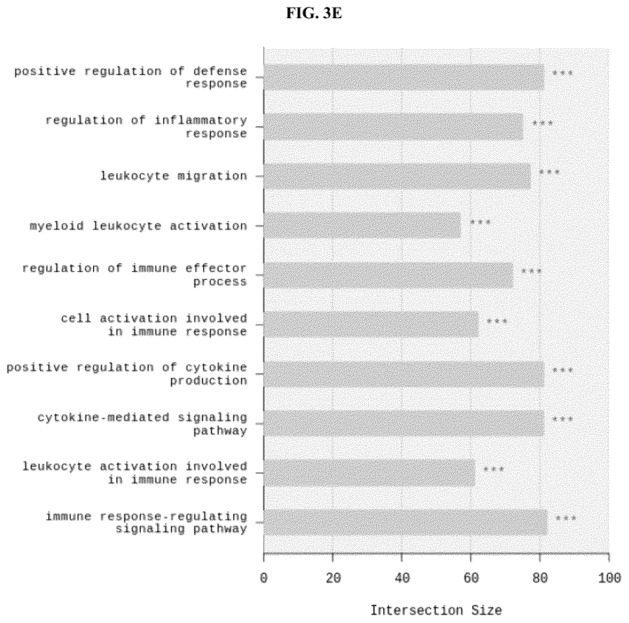

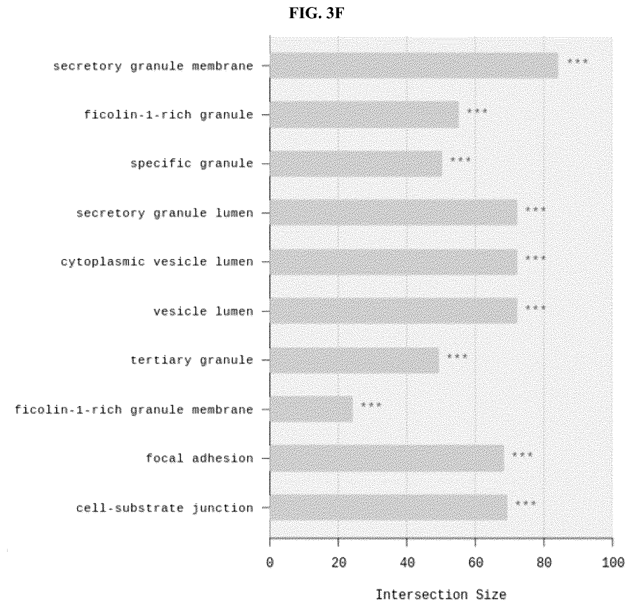

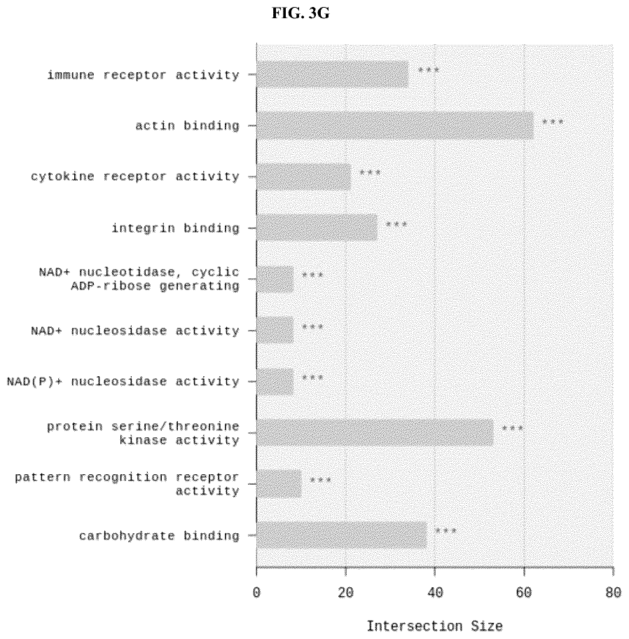

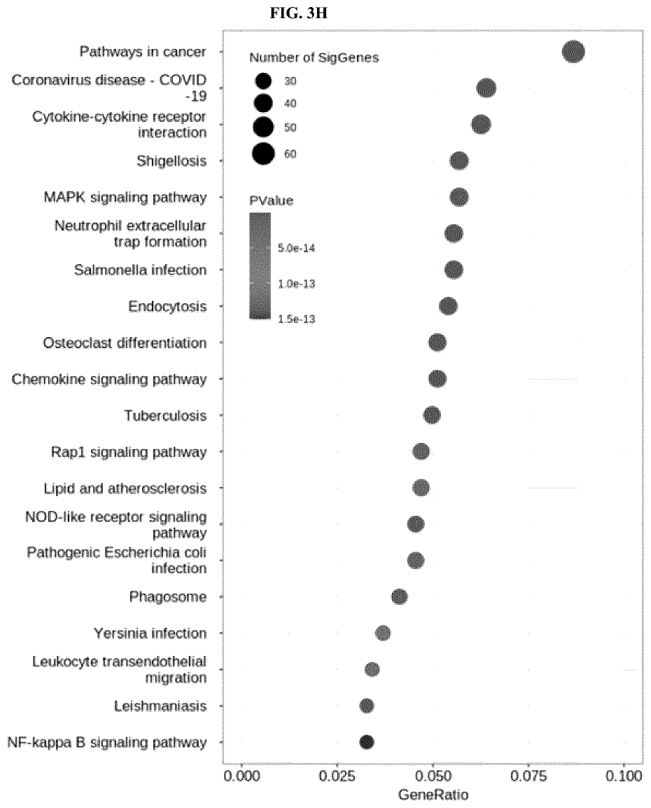

The functional annotation and enrichment analysis results of the DEGs are shown in FIGS. 3A to 3D. Most of the highest terms in the gene ontology (GO) analysis were cytokine-related inflammation, immune responses, cell membrane, or secretory granule-related functions of the SMG tissue (FIGS. 3A to 3C). The KEGG pathway analysis revealed the DEGs interacting with cytokines, inflammation, and immune-related pathways in the SMG tissue (FIG. 3D). Although the GO terms in the blood samples were similar (FIGS. 3E to 3F), viral or other infection-related pathways were the highest interacting pathways in the KEGG analysis (FIG. 3H).

Since the filtering criterion was |case/control fold change|≥2, the expression of SNCA-related genes, which are genes encoding α-synuclein protein and which were filtered out due to differential expression below the threshold, were carefully examined. In particular, SNCA gene expression in the SMG tissue was not significantly different between the PD patients and controls.

However, in the PD patients, SNCAIP gene expression, known as synphilin-1, was significantly down-regulated (fold change=−1.9395, p=0.0371) (Table 3). No significant difference was found in the expression associated with the SNCA gene between the PD patients and the controls in the blood samples.

TABLE 3

| | | | | FDR |
|---|---|---|---|---|
| Gene | | P/C fold | | adjusted |
| symbol | Gene function | change | raw p-value | p-value |
| SNCA | synuclein alpha protein | −1.0826 | 0.5848 | 0.8344 |
| | coding gene | | | |
| SNCAIP | synuclein alpha interacting | −1.9395 | 0.0371 * | 0.2428 |
| | protein coding gene | | | |

### Example 3. Prediction of Functional Interaction Between miRNA and Target Gene

To analyze the functional roles of the differentially expressed miRNAs in the SMG tissue and blood samples from the PD patients, miRbase version 22, which is a miRNA database for miRNA gene annotation, was utilized. Because miRNA induces the degradation or destabilization of mRNA and changes its expression level, a negative correlation is predicted when miRNA-mRNA functionally interact. Predicted mRNA targets of the differentially expressed miRNAs were extracted from the database, and miRNAs exhibiting a negative correlation with actual gene expression in the samples were selected. Through this, the actual regulation of gene expression in the PD patients was confirmed rather than relying only on in silico predictions. The significance of miRNA-mRNA negative correlation pairs was calculated using a hypogeometric test. Significant miRNAs were selected when the p-value was <0.05.

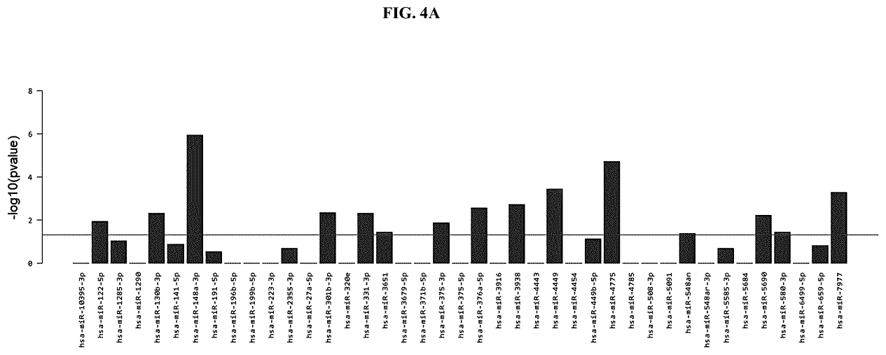

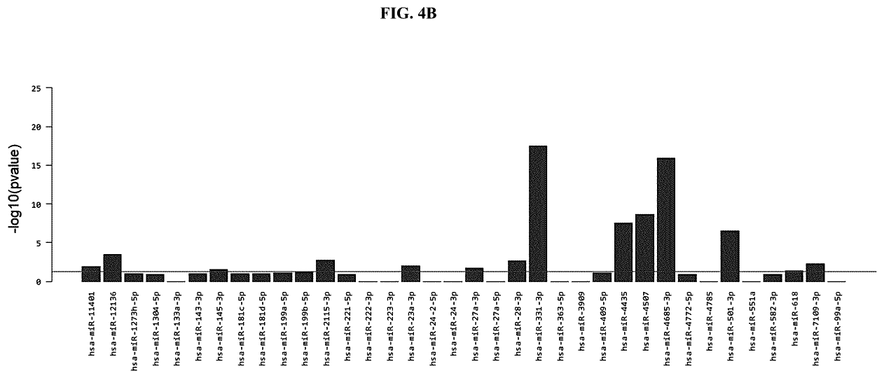

As a result, among the differentially expressed miRNAs in the SMG tissue (41 miRNAs) and the blood (38 miRNAs), 15 significant mature miRNAs were identified in the SMG tissue, and 14 significant mature miRNAs in the blood showed a negative correlation with the target genes expressed in each sample type (FIGS. 1A, 4A, and 4B). Regarding the SNCAIP gene, no miRNA with a significant correlation was detected in the SMG tissue (Table 3, expression of α-synuclein-related genes in SMG) (Table 4, negatively correlated miRNAs targeting SNCAIP) (Table 5, positively correlated miRNAs targeting SNCAIP).

TABLE 4

| | | | FDR adjusted |
|---|---|---|---|
| MicroRNA | P/C fold change | raw p-value | p-value |
| hsa-let-7b-3p | 1.2341 | 0.3422 | 1.0000 |
| hsa-let-7f-2-3p | 1.2565 | 0.3744 | 1.0000 |
| hsa-miR-1185-1-3p | 1.0079 | 1.0000 | 1.0000 |
| hsa-miR-1185-2-3p | 1.1964 | 0.6896 | 1.0000 |
| hsa-miR-130b-5p | 1.4697 | 0.1519 | 1.0000 |
| hsa-miR-138-5p | 1.1919 | 0.4802 | 1.0000 |
| hsa-miR-149-5p | 1.0585 | 0.8055 | 1.0000 |
| hsa-miR-194-3p | 1.4283 | 0.4733 | 1.0000 |
| hsa-miR-2114-5p | 1.0712 | 0.9228 | 1.0000 |
| hsa-miR-224-3p | 1.0657 | 0.8467 | 1.0000 |
| hsa-miR-3064-5p | 1.3038 | 0.6159 | 1.0000 |
| hsa-miR-3085-3p | 1.3674 | 0.7026 | 1.0000 |
| hsa-miR-33a-3p | 1.4519 | 0.1405 | 1.0000 |
| hsa-miR-3661 | 1.7425 | 0.3042 | 1.0000 |
| hsa-miR-4284 | 2.1544 | 0.0890 | 0.9913 |
| hsa-miR-4477b | 1.4581 | 0.6712 | 1.0000 |
| hsa-miR-522-3p | 1.0648 | 0.9385 | 1.0000 |
| hsa-miR-545-3p | 1.1775 | 0.7316 | 1.0000 |
| hsa-miR-548av-3p | 1.3254 | 0.2411 | 1.0000 |
| hsa-miR-548az-5p | 1.2505 | 0.6447 | 1.0000 |
| hsa-miR-548t-5p | 1.5142 | 0.5210 | 1.0000 |
| hsa-miR-5680 | 1.2701 | 0.5155 | 1.0000 |
| hsa-miR-5699-5p | 1.3809 | 0.2628 | 1.0000 |
| hsa-miR-589-3p | 1.8453 | 0.0512 | 0.9913 |
| hsa-miR-597-3p | 1.0801 | 0.8990 | 1.0000 |
| hsa-miR-664a-5p | 1.4142 | 0.1659 | 1.0000 |
| hsa-miR-7974 | 1.3724 | 0.6764 | 1.0000 |
| hsa-miR-93-3p | 1.0412 | 0.8684 | 1.0000 |
| hsa-miR-98-3p | 1.0491 | 0.8216 | 1.0000 |

TABLE 5

| | | | FDR adjusted |
|---|---|---|---|
| MicroRNA | P/C fold change | raw p-value | p-value |
| hsa-let-7a-3p | −1.0738 | 0.7601 | 1.0000 |
| hsa-let-7f-1-3p | −1.0169 | 0.9535 | 1.0000 |
| hsa-miR-12136 | −1.5221 | 0.1922 | 1.0000 |
| hsa-miR-124-3p | −1.2842 | 0.4256 | 1.0000 |
| hsa-miR-133a-5p | −1.0106 | 0.9795 | 1.0000 |
| hsa-miR-153-5p | −1.5234 | 0.2646 | 1.0000 |
| hsa-miR-190a-3p | −1.0907 | 0.7703 | 1.0000 |
| hsa-miR-218-5p | −1.2110 | 0.4186 | 1.0000 |
| hsa-miR-24-3p | −1.4575 | 0.2692 | 1.0000 |
| hsa-miR-3609 | −1.2802 | 0.4467 | 1.0000 |
| hsa-miR-3617-3p | −2.3832 | 0.3210 | 1.0000 |
| hsa-miR-4794 | −1.4226 | 1.0000 | 1.0000 |
| hsa-miR-499a-3p | −1.0137 | 0.9339 | 1.0000 |
| hsa-miR-506-3p | −1.2765 | 0.8187 | 1.0000 |
| hsa-miR-5094 | −1.7292 | 0.7898 | 1.0000 |
| hsa-miR-548ah-5p | −1.0170 | 0.9557 | 1.0000 |
| hsa-miR-6504-5p | −1.9277 | 0.0820 | 0.9913 |
| hsa-miR-6802-3p | −1.0835 | 1.0000 | 1.0000 |
| hsa-miR-6815-5p | −2.5375 | 0.1370 | 1.0000 |
| hsa-miR-934 | −1.6900 | 0.2514 | 1.0000 |

### Example 4. Comparison of Tissue-Specific Transcriptional Regulation and Diagnostic Ability of miRNA Signatures for PD Between SMG and Blood

Among the DEGs and miRNAs in the SMG tissue and blood of the PD patients, the number of common genes and common miRNAs was quantified. Only 73 out of the 533 DEGs in the SMG tissue and 1,776 DEGs in the blood were down-regulated in the PD patients (Table 6). Among the sample types, hsa-miR-331-3p was the only common miRNA, and there were only two genes (THBS1 and NFAM1) regulated by hsa-miR-331-3p in the SMG tissue and blood (Table 7).

TABLE 6

| | SMG | | Common | | Blood | |
|---|---|---|---|---|---|---|
| MicroRNA | P/C FC | raw p-value | gene symbol | P/C FC | raw p-value | MicroRNA |
| hsa-miR-122-5p | −4.725721 | 5.16725E−06 | BCL2A1 | −4.037918 | 0.032645647 | |
| | −2.500100 | 0.046100675 | C4A | −2.695161 | 0.000710347 | |
| | −2.507842 | 0.048949998 | C4B | −2.814209 | 0.000528877 | |
| | −4.931421 | 1.61442E−05 | CEBPD | −3.827470 | 0.000109569 | |
| hsa-miR-5690 | −2.165655 | 1.9909E−05 | CCR1 | −2.987953 | 7.81833E−06 | |
| | −2.265661 | 0.00072202 | PLK3 | −2.403917 | 0.001557917 | |
| hsa-miR-4775 | −3.137577 | 0.000311199 | CR1 | −5.922396 | 9.90477E−08 | hsa-miR-12136 |
| hsa-miR-548ar-3p | −2.031683 | 0.007367019 | CYP1B1 | −4.171870 | 3.83913E−05 | hsa-miR-27a-3p |
| hsa-miR-148a-3p | −4.828940 | 5.56724E−12 | DUSP1 | −3.079049 | 0.000712624 | hsa-miR-12136 |
| | −4.421832 | 2.19734E−21 | GPR183 | 2.288184 | 0.010415659 | hsa-miR-12136 |
| hsa-miR-148a-3p | −4.466603 | 3.27918E−06 | EMP1 | 3.803685 | 3.3972E−06 | hsa-miR-12136 |
| | −3.856283 | 2.78123E−05 | FCGR3B | −2.396745 | 0.004103045 | |
| | −2.533062 | 0.005231838 | FCN1 | −2.042030 | 1.27689E−05 | |
| hsa-miR-548ar-3p | −74.030797 | 2.8016E−23 | FOS | −2.994268 | 0.000771862 | hsa-miR-501-3p |
| | −3.587935 | 2.48549E−06 | FPR1 | −3.202315 | 1.69422E−06 | |
| | −2.559412 | 0.03311366 | CXCL1 | −2.411925 | 0.000244231 | |
| | −2.002988 | 3.50607E−10 | CFH | 2.181260 | 0.020916315 | |
| | −3.847131 | 7.76329E−10 | ICAM1 | −2.276567 | 0.001349 | |
| | −9.396700 | 7.02131E−12 | IL1B | −3.357036 | 1.1889E−05 | |
| hsa-miR-4449 | −2.187314 | 0.004811917 | IL7R | 2.440283 | 1.18016E−05 | |
| hsa-miR-5690 | −2.255802 | 0.004278587 | ITGB3 | −2.613583 | 0.003007794 | |
| hsa-miR-376a-5p | −4.962596 | 1.04814E−08 | JUNB | −3.759492 | 4.13784E−06 | hsa-miR-11401, |
| | | | | | | hsa-miR-12136 |
| | −2.890121 | 5.76249E−06 | GADD45B | −2.746131 | 4.26707E−05 | hsa-miR-4507 |
| | −2.130678 | 0.022363974 | NFIL3 | −3.113215 | 0.000385667 | hsa-miR-27a-3p |
| | −2.078298 | 0.024014698 | CFP | −2.239263 | 0.000141207 | |
| hsa-miR-4775 | −2.384281 | 2.66726E−08 | SRGN | −2.150202 | 0.0416045 | |
| | −3.310554 | 0.000371783 | S100A9 | −3.865474 | 0.00027415 | |
| | −3.378971 | 5.70437E−05 | SELL | −2.795192 | 0.000296918 | |
| hsa-miR-580-3p | −2.569680 | 9.85173E−06 | SELP | −2.092733 | 0.001719843 | |
| | −2.098304 | 0.000247792 | SLA | −2.703023 | 0.000225285 | |
| hsa-miR-122-5p | −5.597034 | 1.81362E−20 | SLC2A3 | −4.644082 | 1.85642E−07 | hsa-miR-4435, |
| | | | | | | hsa-miR-4685-3p |
| hsa-miR-7977 | −2.556416 | 0.004770806 | SLC11A1 | −3.746816 | 2.15278E−05 | |
| | −2.022463 | 0.003260985 | THBD | −3.830043 | 0.000103441 | |
| hsa-miR-331-3p | −2.279068 | 0.002140368 | THBS1 | −3.114932 | 1.09916E−07 | hsa-miR-331-3p, |
| | | | | | | hsa-miR-4685-3p, |
| | | | | | | hsa-miR-618 |
| | −9.053542 | 6.55305E−12 | ZFP36 | −3.072648 | 7.32945E−05 | hsa-miR-27a-3p |
| hsa-miR-548an | −2.636232 | 2.83112E−09 | BTG2 | −2.590598 | 0.000344713 | hsa-miR-27a-3p |
| | −2.168830 | 3.48997E−06 | DYSF | −6.910059 | 3.15265E−07 | |
| | −2.762634 | 0.000410067 | CST7 | −4.052690 | 3.37603E−05 | |
| hsa-miR-548an | −2.024839 | 0.001303256 | STX11 | −3.499122 | 5.00642E−05 | hsa-miR-12136, |
| | | | | | | hsa-miR-27a-3p |
| | −2.072427 | 0.021208686 | ABCC3 | −2.862026 | 0.005561681 | |
| | −2.157484 | 0.001463343 | IER3 | −2.335862 | 0.010406582 | |
| hsa-miR-3651 | −3.266901 | 0.000642355 | VNN2 | −3.533973 | 0.001575622 | |
| hsa-miR-122-5p | −2.329885 | 0.009914473 | VNN1 | −3.397833 | 0.000498871 | |
| hsa-miR-4449 | −20.900030 | 5.81655E−12 | SOCS3 | −15.072286 | 5.32784E−12 | |
| hsa-miR-4775 | −2.352986 | 0.00020581 | CD163 | −3.927915 | 7.18413E−06 | |
| | −3.253796 | 2.99739E−11 | PIM2 | −2.244494 | 0.00061217 | |
| | −3.213837 | 2.08809E−07 | PPP1R15A | −2.304034 | 0.000147278 | |
| | −2.090438 | 0.002033675 | IGLV3-25 | −2.314552 | 0.04315051 | |
| hsa-miR-376a-5p | −4.172894 | 8.61063E−07 | ICOS | 2.153583 | 0.000494472 | hsa-miR-27a-3p |
| hsa-miR-3651 | −2.331547 | 0.024337291 | TLR8 | −2.562349 | 0.000284667 | |
| | −2.193972 | 0.000150465 | SAMSN1 | −3.219301 | 0.002421553 | |
| hsa-miR-3938 | −2.142919 | 5.29026E−09 | NFKBIZ | −2.611214 | 3.28634E−05 | |
| hsa-miR-301b-3p | −2.245937 | 4.41178E−08 | NABP1 | −2.108490 | 0.001890567 | hsa-miR-27a-3p |
| hsa-miR-301b-3p | −2.137579 | 3.77876E−05 | MCTP1 | −2.050468 | 0.002574684 | hsa-miR-12136 |
| | −2.074873 | 6.73903E−06 | EMILIN2 | −2.652938 | 4.91549E−05 | |
| | −2.101016 | 6.26058E−06 | ITPRIP | −2.263424 | 0.00051204 | hsa-miR-618 |
| | −2.194508 | 2.96328E−05 | NLRP3 | −3.013511 | 5.54862E−05 | |
| | −2.574238 | 0.009266354 | ANKRD22 | −4.432498 | 0.001936942 | |
| hsa-miR-7977 | −2.760564 | 0.000221501 | JAML | −2.087735 | 0.001211197 | |
| hsa-miR-4775 | −2.083408 | 0.0263416 | TRABD2A | 2.225428 | 0.002767707 | hsa-miR-4507 |
| | −2.089149 | 0.010671064 | BTBD19 | −3.007833 | 0.001155173 | |
| hsa-miR-331-3p | −2.375153 | 0.009259148 | NFAM1 | −3.754747 | 0.000448128 | hsa-miR-331-3p |
| | 4.214078 | 0.004541735 | RPL10P16 | 5.442146 | 0.000138108 | |
| | 2.602471 | 0.019087529 | RPL29P4 | 3.295480 | 0.001039792 | |
| | 68.609766 | 2.5347E−06 | RPL10P9 | 118.708800 | 5.73245E−07 | |
| | −2.329862 | 0.015891934 | LOC441081 | −9.864287 | 3.33231E−07 | |
| | −2.390754 | 0.000298435 | NAMPTP1 | −2.863294 | 0.007482772 | |
| | −2.246858 | 0.003380982 | NCF1 | −2.204434 | 0.001291324 | |
| | −2.656574 | 0.000217669 | NCF1B | −2.042988 | 0.003796592 | |
| | −2.152064 | 0.021475055 | FCGR1CP | −5.279120 | 1.88105E−05 | |
| | −3.557532 | 6.59875E−05 | RNVU1-14 | 2.335143 | 0.01122683 | |
| | −2.042709 | 0.010264351 | LOC102723360 | −2.209455 | 0.028875165 | hsa-miR-12136 |
| hsa-miR-122-5p | −2.321557 | 0.004300152 | LOC107986613 | 2.314788 | 0.012711799 | |

TABLE 7

| Targeted | SMG | | Common | Targeted | Blood | |
|---|---|---|---|---|---|---|
| gene symbol | P/C FC | raw p-value | microRNA | gene symbol | P/C FC | raw p-value |
| DUSP5 | −4.801738 | 9.54052E−07 | hsa-miR-331-3p | AQP9 | −3.584254 | 4.2873E−05 |
| FGF11 | −2.104888 | 0.010943892 | | CNN2 | −2.667414 | 0.004120064 |
| CXCL9 | −2.541261 | 0.039536466 | | CUX1 | −2.409842 | 0.001050514 |
| PIM1 | −2.121084 | 9.00782E−05 | | ARID3A | −3.136860 | 6.43477E−05 |
| THBS1 * | −2.279068 | 0.002140368 | | PHC2 | −3.756023 | 0.00029434 |
| KCNE4 | −2.117927 | 0.007926481 | | GNAZ | −2.071207 | 0.023431834 |
| NFAM1 * | −2.375153 | 0.009259148 | | LSP1 | −2.844348 | 2.88906E−05 |
| EPOP | 3.064727 | 0.001560926 | | MAFG | −3.255636 | 6.99245E−06 |
| | | | | MNT | −2.899753 | 0.000656958 |
| | | | | PML | −2.328198 | 0.024684715 |
| | | | | NECTIN1 | −2.829434 | 0.001030185 |
| | | | | SLC9A1 | −3.098512 | 0.00354239 |
| | | | | SOD2 | −2.453118 | 0.00046603 |
| | | | | THBS1 * | −3.114932 | 1.09916E−07 |
| | | | | GAS7 | −3.964507 | 1.26544E−05 |
| | | | | ADAM19 | −3.032268 | 8.91256E−05 |
| | | | | H6PD | −2.345889 | 0.001461068 |
| | | | | TBKBP1 | −2.860069 | 0.004664854 |
| | | | | HDAC5 | −2.061604 | 0.004932731 |
| | | | | RNF44 | −2.159634 | 0.026680541 |
| | | | | PHLPP1 | −2.411466 | 0.004701039 |
| | | | | ANKS1A | −2.027446 | 0.004719255 |
| | | | | CDC42EP4 | −2.518654 | 0.00532939 |
| | | | | TBC1D10B | −2.871798 | 0.001645804 |
| | | | | MINK1 | −2.020686 | 0.028911833 |
| | | | | C14orf132 | 3.126097 | 0.00356986 |
| | | | | TMCC3 | −2.046601 | 0.008267345 |
| | | | | SLC13A3 | 4.232253 | 0.005737561 |
| | | | | CLMN | −2.035627 | 0.002072337 |
| | | | | RAB1B | −2.323708 | 0.006393768 |
| | | | | KLF16 | −2.545196 | 0.006686909 |
| | | | | NACC1 | −2.391548 | 0.013145289 |
| | | | | MBD6 | −3.183199 | 0.004316468 |
| | | | | LOXHD1 | −3.430097 | 0.000180801 |
| | | | | NFAM1 * | −3.754747 | 0.000448128 |
| | | | | MSL1 | −2.244646 | 0.000582577 |
| | | | | ZC3H12B | 2.662456 | 0.000163893 |
| | | | | SAMD12 | 2.558944 | 0.002293958 |

Because the miRNA signature was unique to each tissue in each sample type of the PD patients, the discriminatory power of the miRNA signatures in the SMG tissue and blood samples was quantified and compared to identify PD patients. In addition, the increase or decrease of each miRNA was confirmed.

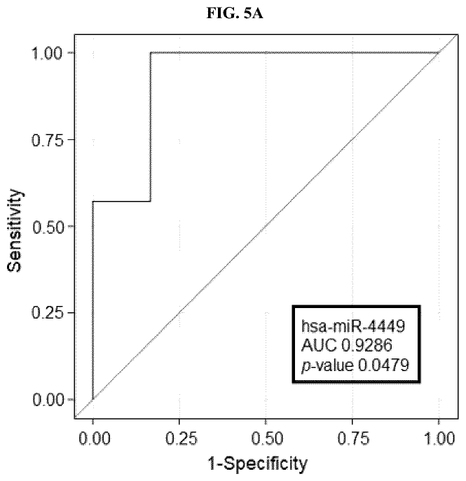

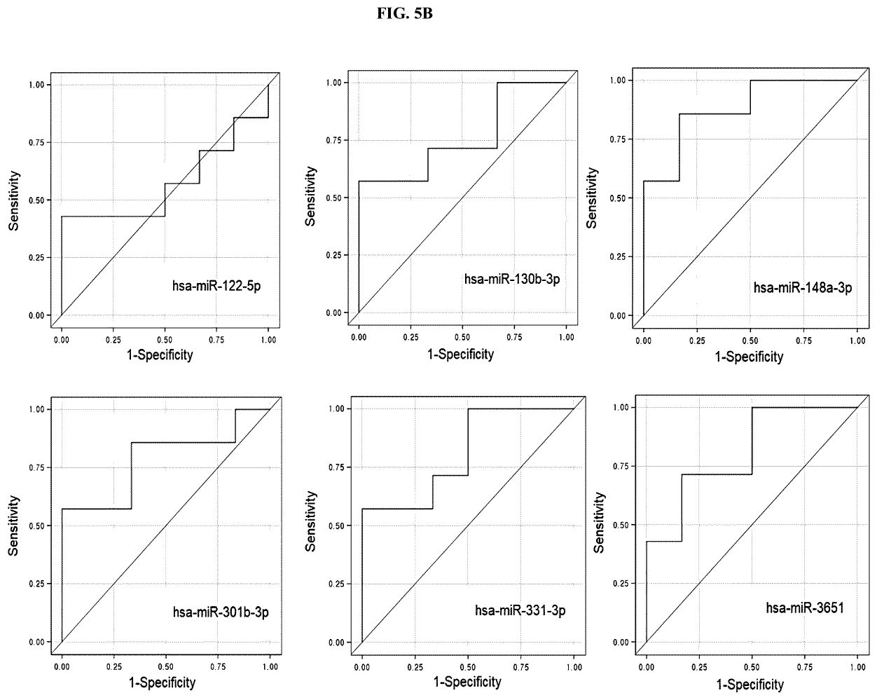

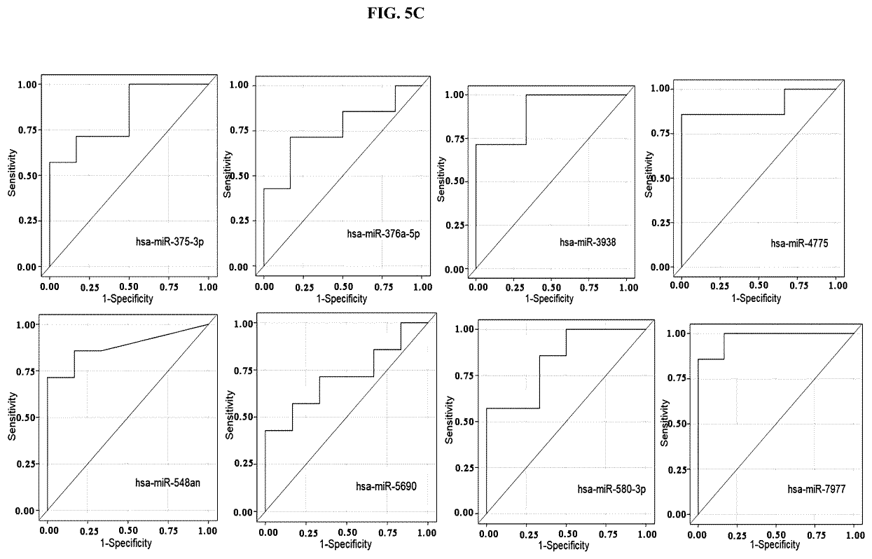

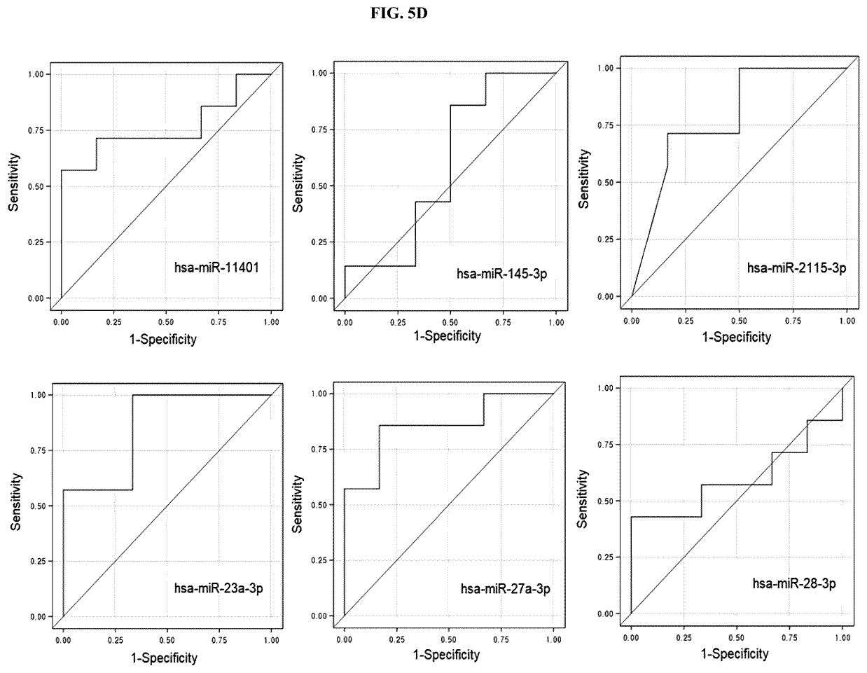

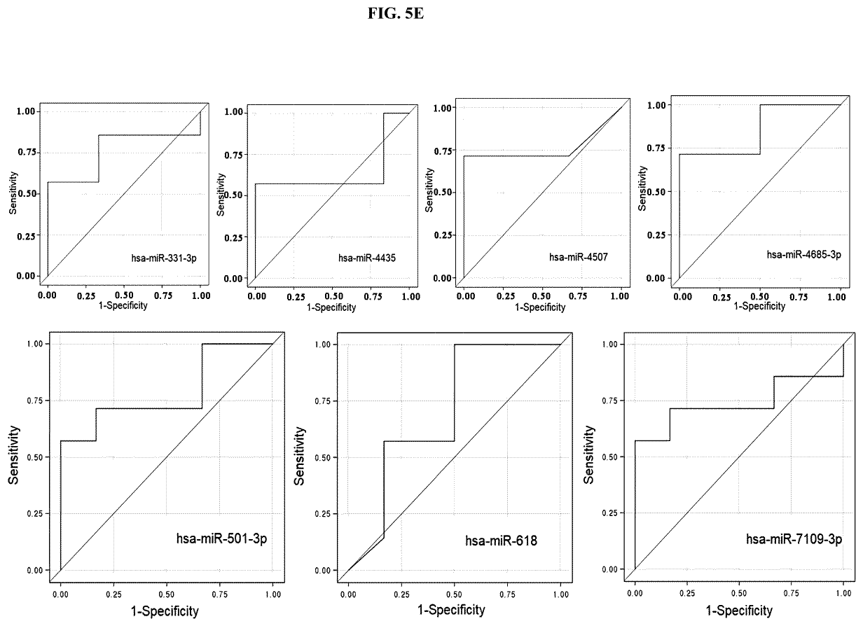

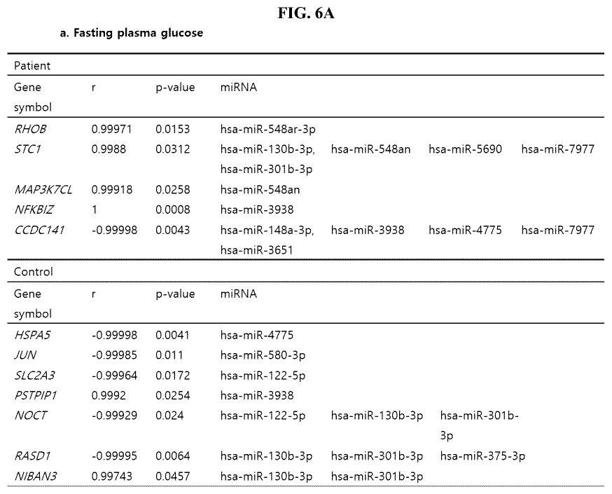

The results are shown in Tables 8 and 9 below. In addition, the ROC curve of each miRNA was confirmed as shown in FIGS. 5B to 5E.

TABLE 8

| | MicroRNAs | Patient/Control | | 95% Wald | | |
|---|---|---|---|---|---|---|
| No. | in SMG | fold change | AUC | Confidence Limits | | p-value |
| 1 | hsa-miR-122-5p | 5.920657 | 0.5714 | 0.008 | >999.999 | 0.5642 |
| 2 | hsa-miR-130b-3p | 2.004716 | 0.7619 | 0.574 | 225.478 | 0.1105 |
| 3 | hsa-miR-148a-3p | 2.490109 | 0.8910 | 0.859 | 42.573 | 0.0706 |
| 4 | hsa-miR-301b-3p | 2.642414 | 0.7857 | 0.751 | 7.288 | 0.1427 |
| 5 | hsa-miR-331-3p | 2.265236 | 0.8095 | 0.689 | 18.813 | 0.1288 |
| 6 | hsa-miR-3651 | 3.816909 | 0.8095 | 0.539 | >999.999 | 0.0927 |
| 7 | hsa-miR-375-3p | 2.208887 | 0.8333 | 0.859 | 18.441 | 0.0775 |
| 8 | hsa-miR-376a-5p | 2.001365 | 0.7619 | 0.355 | 670.138 | 0.1550 |
| 9 | hsa-miR-3938 | 3.319845 | 0.9048 | 0.034 | >999.999 | 0.0873 |
| 10 | hsa-miR-4449 | 2.791364 | 0.9286 | 1.064 | >999.999 | 0.0479* |
| 11 | hsa-miR-4775 | 2.478404 | 0.9048 | 0.808 | >999.999 | 0.0604 |
| 12 | hsa-miR-548an | 3.884255 | 0.8910 | 0.414 | >999.999 | 0.0570 |
| 13 | hsa-miR-5690 | 2.319359 | 0.7143 | 0.248 | 274.241 | 0.2379 |
| 14 | hsa-miR-580-3p | 2.620176 | 0.8333 | 0.708 | 216.637 | 0.0849 |
| 15 | hsa-miR-7977 | 2.245683 | 0.9762 | 0.101 | >999.999 | 0.1413 |

TABLE 9

| | MicroRNAs | Patient/Control | | 95% Wald | | |
|---|---|---|---|---|---|---|
| No. | in blood | fold change | AUC | Confidence Limits | | p-value |
| 1 | hsa-miR-11401 | 3.077674 | 0.7619 | 0.749 | 14.132 | 0.1156 |
| 2 | hsa-miR-12136 | −5.25138 | — | — | — | — |
| 3 | hsa-miR-145-3p | −5.13289 | 0.5952 | 0.002 | 13.057 | 0.3965 |
| 4 | hsa-miR-2115-3p | −6.30624 | 0.7857 | <0.001 | 808.235 | 0.1175 |
| 5 | hsa-miR-23a-3p | −2.54386 | 0.8571 | 0.028 | 1.099 | 0.0631 |
| 6 | hsa-miR-27a-3p | −2.5836 | 0.8571 | 0.035 | 1.071 | 0.0600 |
| 7 | hsa-miR-28-3p | 3.234461 | 0.5952 | 0.720 | 2.727 | 0.3203 |
| 8 | hsa-miR-331-3p | 2.591601 | 0.7619 | 0.732 | 11.583 | 0.1293 |
| 9 | hsa-miR-4435 | 3.771794 | 0.6429 | 0.671 | 12.763 | 0.1529 |
| 10 | hsa-miR-4507 | 4.633707 | 0.7619 | <0.001 | >999.999 | 0.1692 |
| 11 | hsa-miR-4685-3p | 2.569675 | 0.8571 | 0.860 | 25.000 | 0.0744 |
| 12 | hsa-miR-501-3p | 2.798923 | 0.7857 | 0.301 | 162.756 | 0.2253 |
| 13 | hsa-miR-618 | −6.1736 | 0.7024 | 0.022 | 3.240 | 0.3004 |
| 14 | hsa-miR-7109-3p | 4.31043 | 0.7381 | 0.004 | >999.999 | 0.2022 |

It was found that the miRNA signatures in the SMG tissue have significantly better discriminatory power in identifying PD patients (mean±SD of AUC; 0.8251±0.1006 and 0.7463±0.0908 in SMG tissue, two-sided p=0.0314).

The highest AUC value in the SMG tissue was for has-miR-4449 (AUC=0.9286, p=0.0479). FIG. 5A shows the ROC curve and AUC value of has-miR-4449. The data supports the SMG tissue as a better sample choice for including miRNA signatures representing PD-related pathophysiology.

### Example 5. Confirmation of Clinical Correlation of Modified Gene Expression in PD Patients

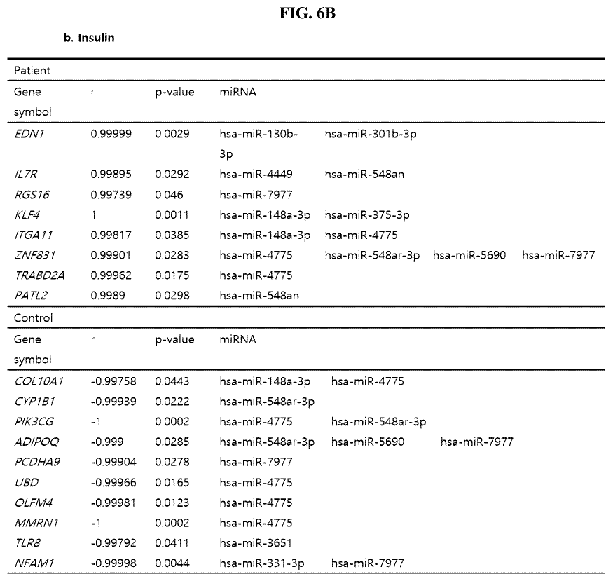

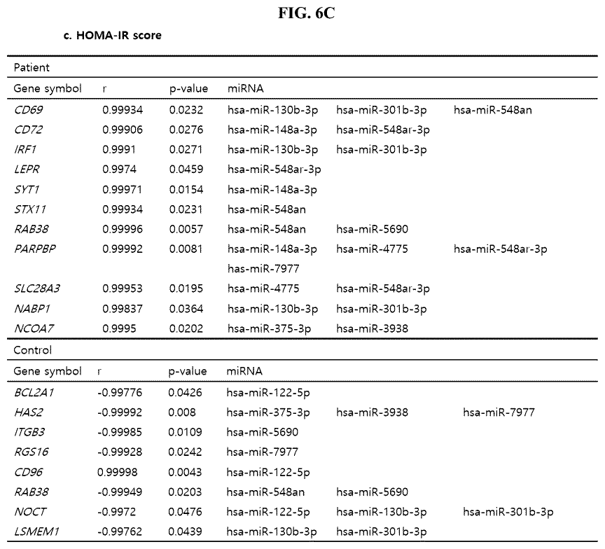

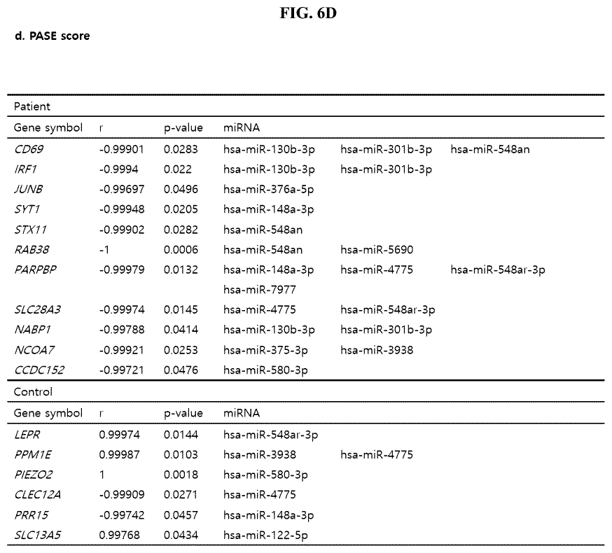

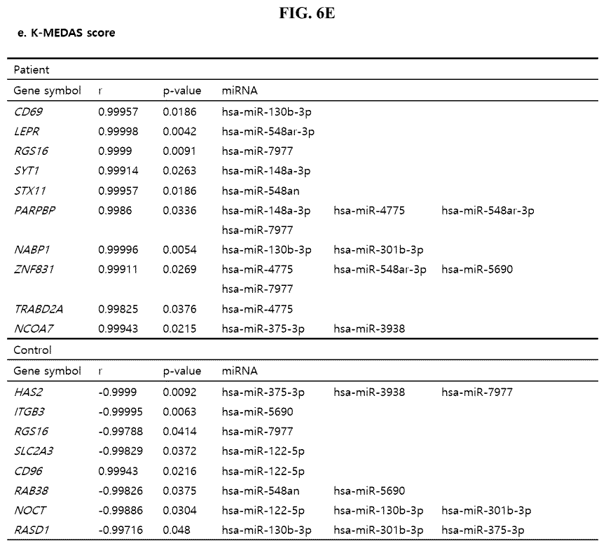

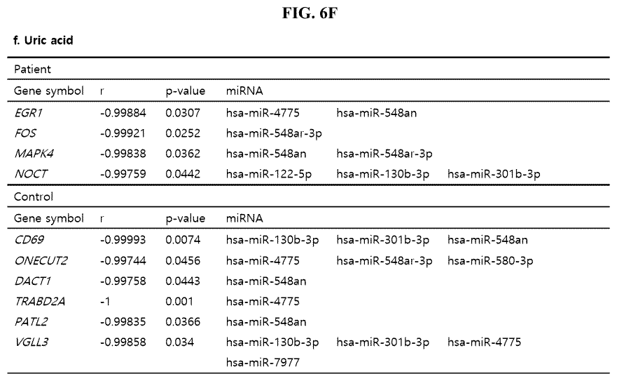

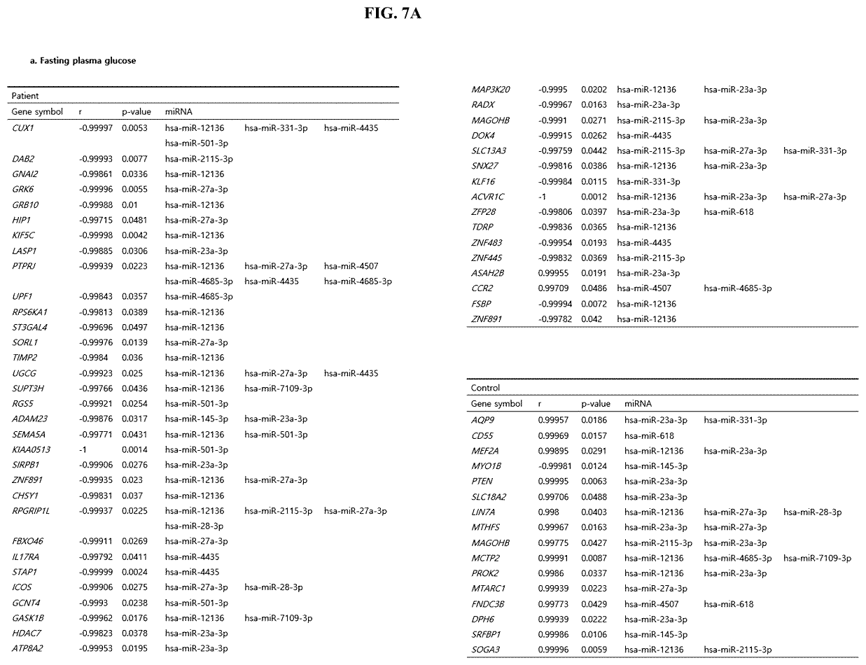

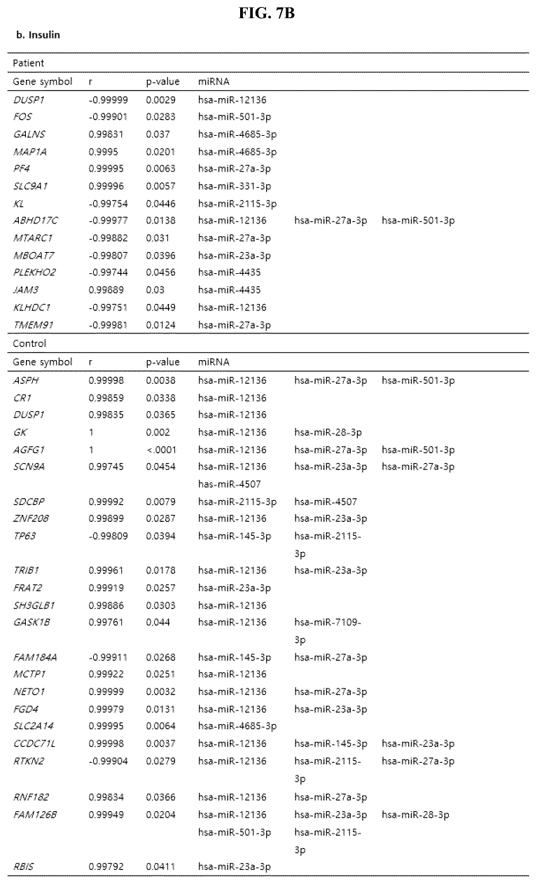

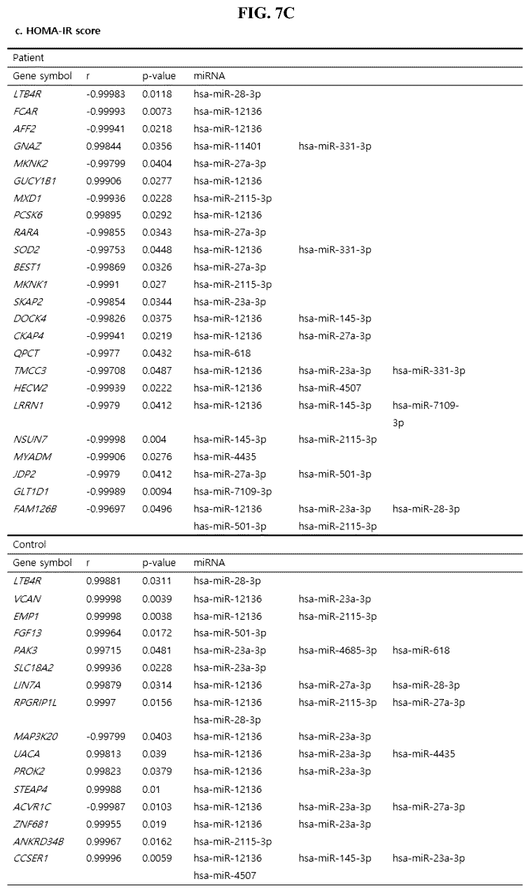

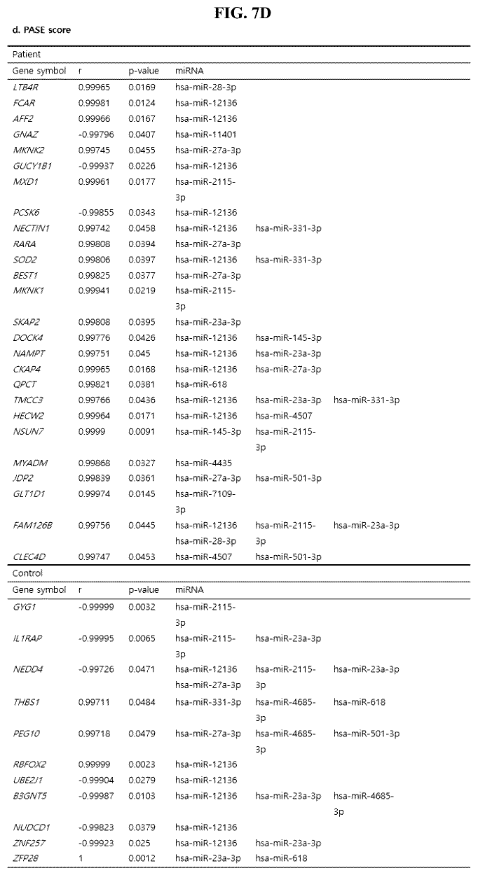

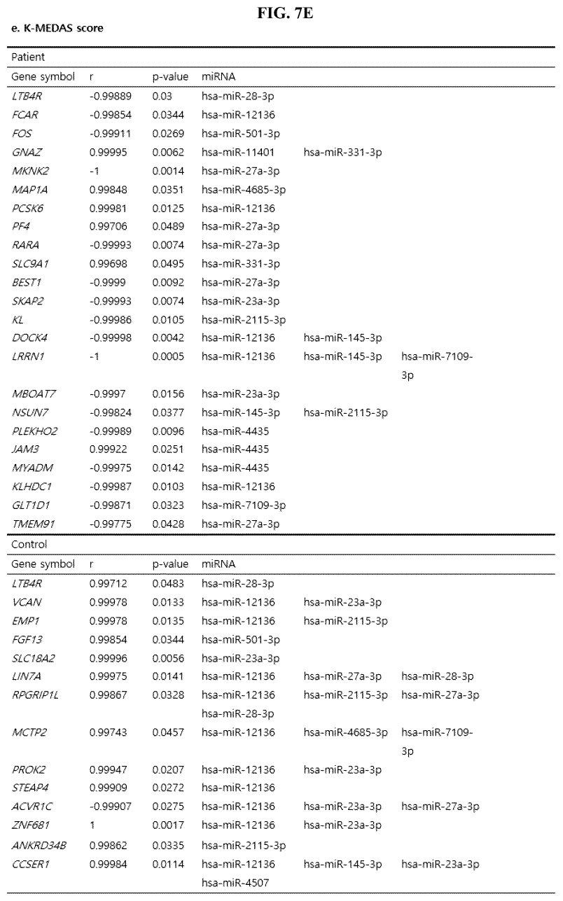

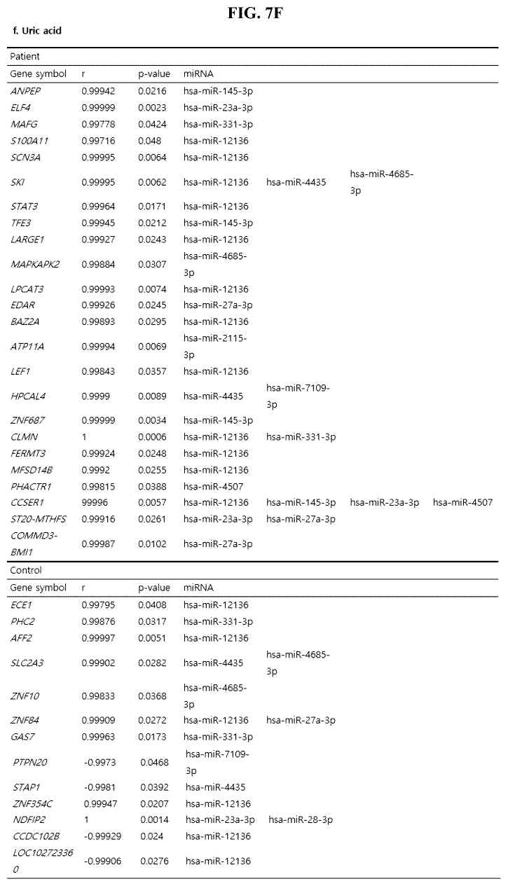

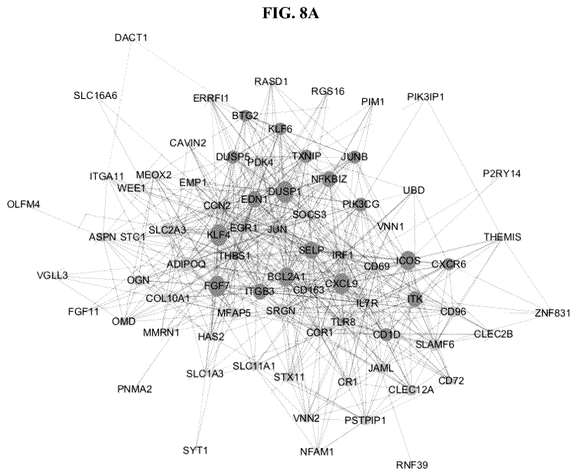

Changes in gene expression without DNA modification may occur due to environmental factors such as diet or physical activity. Therefore, the correlations of the DEGs of the PD patients with clinical factors that are known to affect the prognosis of PD were evaluated, including fasting plasma glucose, serum insulin, insulin resistance (HOMA-IR score), uric acid, dietary pattern (K-MEDAS score), and physical activity (PASE score). DEGs with a significant partial correlation coefficient (r) and p-value are shown in FIGS. 6A to 6F for the SMG tissue and in FIGS. 7A to 7F for the blood samples.

In particular, the same clinical factors affected different DEGs in the PD patients compared to the controls, and the same genes in the PD patients were influenced by different clinical factors compared to the controls. For example, leptin receptor gene (LEPR) expression had a positive correlation with the HOMA-IR score and the dietary score in the PD patients. However, the clinical factor that had a positive correlation with LEPR gene expression in the SMG tissue in the control group was the physical activity score (FIGS. 6A to 6F). A greater diversity of DEGs in the blood samples were correlated with these environmental factors, but the environmental influences on gene expression were also altered in the PD patients compared to the blood samples of the controls (FIGS. 7A to 7F).

### Example 6. Confirmation of the Interaction Network of Target Genes with DEmiRNAs in SMG or Blood

In the living body, two or more proteins interact to form a highly complex network and tightly controlled protein interactions underlie nearly all life phenomena. Accordingly, in the present invention, the protein-protein interaction (PPI) network was confirmed using the Search Tool for Interacting Genes (STRING) database with a confidence threshold of 0.2.

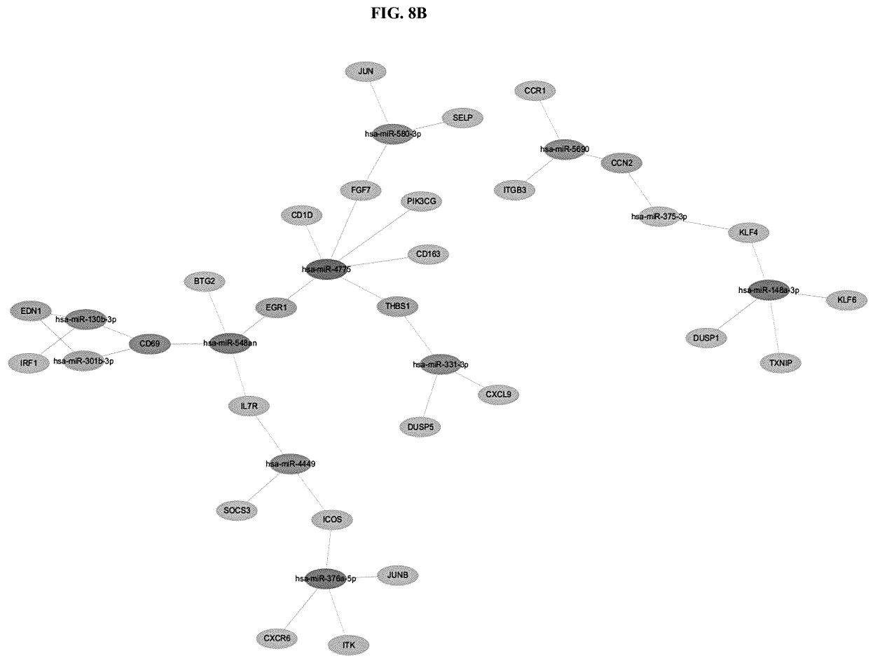

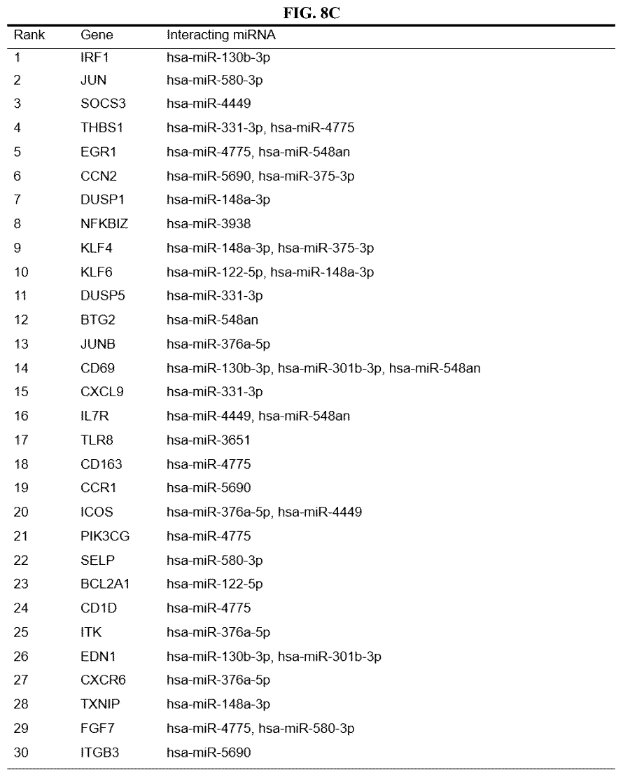

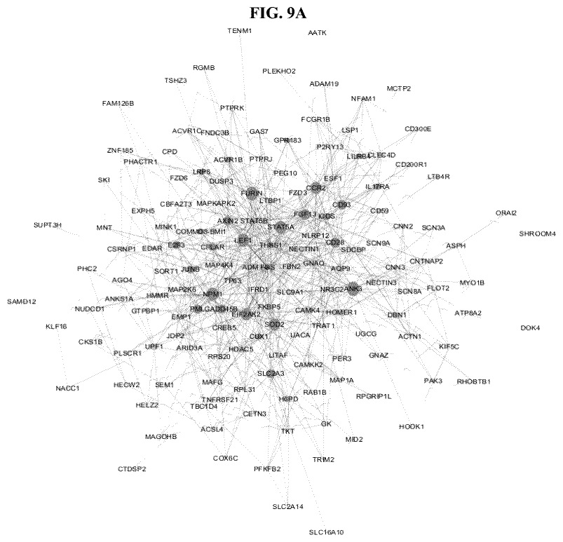

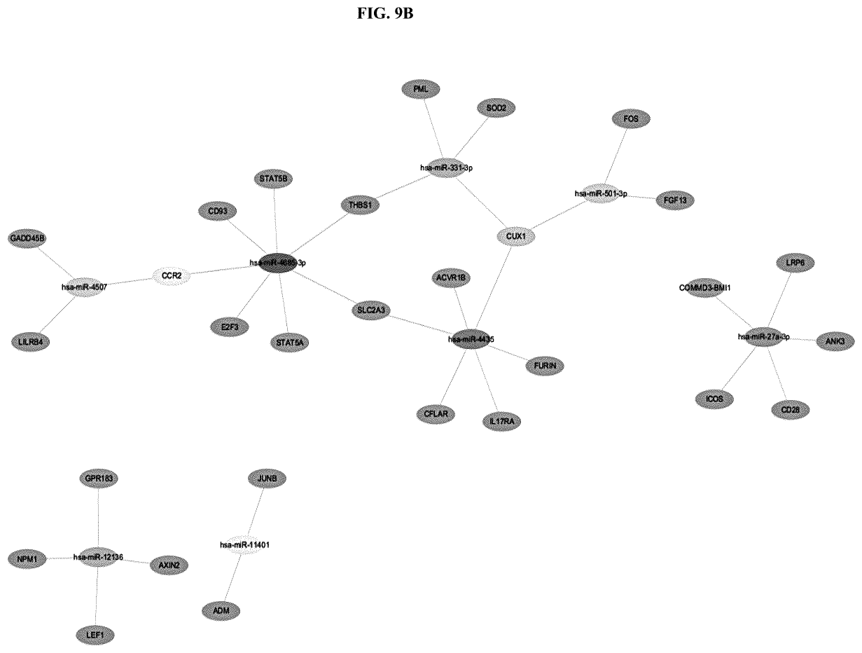

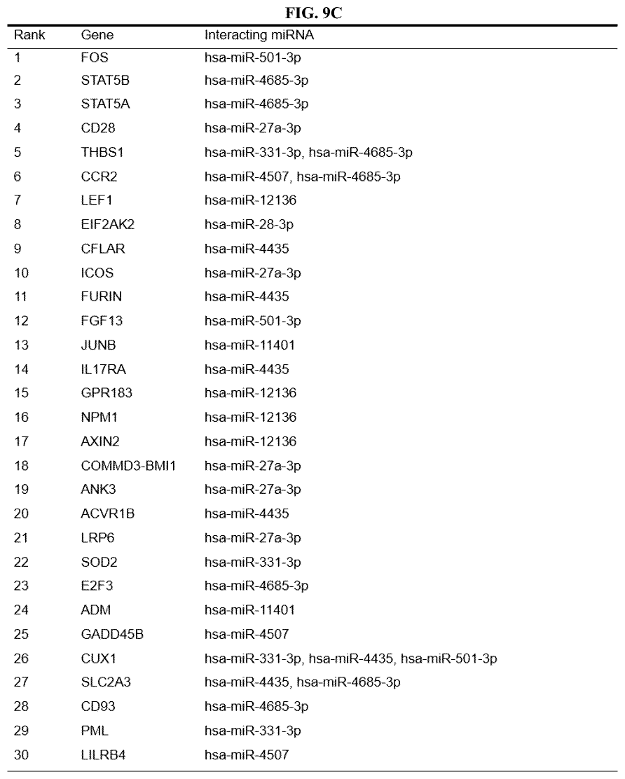

As a result. The interaction network of target genes and differentially expressed miRNAs (DEmiRNAs) identified by protein-protein interaction network analysis showed up-down regulation in SMG (FIGS. 8A and 8B), and either up-down or down-up regulation in blood (FIGS. 9A and 9B). Among these, 11 DEmiRNAs interacted with the top 30 hub genes in SMG (FIG. 8C), while 8 DEmiRNAs interacted with the top 30 hub genes in blood (FIG. 9C).

The description of the present invention described above is for illustrative purposes, and those skilled in the art will understand that the present invention can be easily modified into other specific forms without changing the technical ideas or essential features of the present invention. Therefore, the embodiments described above should be understood as illustrative in all respects and not restrictive.

What is claimed is:

1. A method comprising the steps of:
(S1) measuring the level of mRNA of an α-synuclein interacting protein (SNCAIP) gene encoding synphilin-1 in a biological sample isolated from a subject;
(S2) comparing the level of the mRNA with the level of the mRNA in a biological sample isolated from a normal control group;
(S3) determining that the subject has Parkinson's disease (PD) when the level of the mRNA in the biological sample isolated from the subject is decreased compared to the level of the mRNA in the biological sample isolated from the control group; and
(S4) treating the PD with chemotherapy, surgery, or biological therapy when PD is diagnosed in step (S3).

2. The method of claim 1, wherein the biological sample is one selected from the group consisting of submandibular gland tissue, blood, serum, whole blood, plasma, urine, saliva, tissue, cells, organs, bone marrow, a fine needle aspiration specimen, a core needle biopsy specimen, and a vacuum aspiration biopsy specimen.

3. A method comprising the steps of:
(S1) measuring the level of any one or more of miRNAs selected from the group consisting of hsa-miR-122-5p, hsa-miR-130b-3p, hsa-miR-148a-3p, hsa-miR-301b-3p, hsa-miR-331-3p, hsa-miR-3651, hsa-miR-375-3p, hsa-miR-376a-5p, hsa-miR-3938, hsa-miR-4449, hsa-miR-4775, hsa-miR-548an, hsa-miR-5690, hsa-miR-580-3p, and hsa-miR-7977 in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is increased compared to the level of the miRNA in the biological sample isolated from the control group; and
(S4) treating the PD with chemotherapy, surgery, or biological therapy when PD is diagnosed in step (S3).

4. The method of claim 3, wherein the biological sample is one selected from the group consisting of submandibular gland tissue, blood, serum, whole blood, plasma, urine, saliva, tissue, cells, organs, bone marrow, a fine needle aspiration specimen, a core needle biopsy specimen, and a vacuum aspiration biopsy specimen.

5. A method comprising the steps of:
(S1) measuring the level of any one or more of miRNAs selected from the group consisting of hsa-miR-130b-3p, hsa-miR-148a-3p, hsa-miR-301b-3p, hsa-miR-331-3p, hsa-miR-375-3p, hsa-miR-376a-5p, hsa-miR-5690, and hsa-miR-580-3p in a biological sample isolated from a subject;
(S2) comparing the level of the miRNA with the level of the miRNA in a biological sample isolated from a normal control group;
(S3) diagnosing that the subject has Parkinson's disease (PD) when the level of the miRNA in the biological sample isolated from the subject is more than 2-fold change increased compared to the level of the miRNA in the biological sample isolated from the control group, and the AUC value is ≥0.5, the p-value is <0.5, the Confidence Limits value falls between 0.001 and 671; and
(S4) treating the PD with chemotherapy, surgery, or biological therapy when PD is diagnosed in step (S3).
